(19) United States
(12) Patent Application Publication
(10) Pub. No.: US 2024/0058476 A1
(43) Pub. Date: Feb. 22, 2024

(54) TREATMENT OF LIPODYSTROPHY

(71) Applicant: THE UNIVERSITY COURT OF THE UNIVERSITY OF ABERDEEN, Aberdeenshire (GB)

(72) Inventors: George David Mcilroy, Aberdeenshire (GB); Justin James Rochford, Aberdeenshire (GB)

(21) Appl. No.: 18/271,404

(22) PCT Filed: Jan. 10, 2022
(86) PCT No.: PCT/EP2022/050358

§ 371 Date: Jul. 7, 2023

(30) Foreign Application Priority Data
Jan. 11, 2021 (GB) 2100311.6

(51) Int. Cl. A61K 48/00; A61P 3/00; C12N 15/86

(52) U.S. Cl. CPC A61K 48/0058; A61P 3/00; C12N 15/86; C12N 2750/14143; C12N 2750/14171

(57) ABSTRACT

This invention relates to the restoration of adipose tissue inpatients with lipodystrophy characterised by a defective gene. A heterologous nucleic acid that encodes a therapeutic gene product is administered to the patient. The therapeutic gene product may be functional version of the protein encoded by the defective gene or an RNA molecule that inhibits expression from the defective gene. Methods for the treatment of lipodystrophy and the amelioration of metabolic dysfunction associated with lipodystrophy are provided, along with agents and compositions for use in such methods.

### PRIORITY

This application claims the benefit of PCT Application No. PCT/EP2022/050358, filed Jan. 10, 2022, which claims the benefit of GB Application No. 2100311.6, filed Jan. 11, 2021, which application are incorporated herein by reference in their entirety.

### INCORPORATION BY REFERENCE OF SEQUENCE LISTING

A Sequence Listing is provided herewith as a Sequence Listing text, MEWE-119_PCTEP2022050358_ST25 created on Jul. 6, 2023, and having a size of 14,023,601 bytes. The contents of the Sequence Listing text are incorporated herein by reference in their entirety.

### FIELD

This invention relates to the treatment of lipodystrophy, in particular the treatment of lipodystrophy using adenovirus associated virus (AAV) mediated gene therapy.

### BACKGROUND

Lipodystrophy is a condition in which adipose tissue is not generated or maintained appropriately in a patient. Adipose tissue normally provides storage for lipids within the body. If adipose tissue is insufficient or absent in a patient, then lipid accumulates in other tissues, including the liver, vasculature and pancreas. This accumulation causes dysfunction of these tissues, leading to metabolic disease. Patients with lipodystrophy typically develop serious metabolic diseases, such as diabetes and fatty liver disease.

### SUMMARY

The present inventors have discovered that viral mediated gene therapy can be used to restore adipose tissue in lipodystrophy patients. This may be useful for example, in the treatment of lipodystrophy and the amelioration of metabolic dysfunction associated with lipodystrophy.

A first aspect of the invention provides a method of treatment of lipodystrophy comprising;
administering to an individual in need thereof a heterologous nucleic acid that encodes a therapeutic gene product,
wherein the lipodystrophy is characterised by a defective gene and the therapeutic gene product encoded by the heterologous nucleic acid is a functional version of the protein encoded by the defective gene or an RNA molecule that inhibits expression from the defective gene.

A second aspect of the invention provides a method of ameliorating metabolic dysfunction in an individual with lipodystrophy comprising;
administering to the individual a heterologous nucleic acid encoding a therapeutic gene product,
wherein the lipodystrophy is characterised by a defective gene and the therapeutic gene product encoded by the heterologous nucleic acid is a functional version of the protein encoded by the defective gene or an RNA molecule that inhibits expression from the defective gene.

A third aspect of the invention provides a heterologous nucleic acid that encodes a therapeutic gene product, for use in a method of treatment of lipodystrophy or a method of ameliorating metabolic dysfunction in an individual with lipodystrophy, wherein the lipodystrophy is characterised by a defective gene and the therapeutic gene product encoded by the heterologous nucleic acid is a functional version of the protein encoded by the defective gene or an RNA molecule that inhibits expression from the defective gene. Suitable methods of treatment of lipodystrophy include methods of the first aspect and suitable methods of ameliorating metabolic dysfunction include methods of the second aspect.

A fourth aspect of the invention provides the use of a heterologous nucleic acid that encodes a therapeutic gene product in the manufacture of a medicament for use in a method of treatment of lipodystrophy or a method of ameliorating metabolic dysfunction in an individual with lipodystrophy, wherein the lipodystrophy is characterised by a defective gene and the therapeutic gene product encoded by the heterologous nucleic acid is a functional version of the protein encoded by the defective gene or an RNA molecule that inhibits expression from the defective gene. Suitable methods of treatment of lipodystrophy include methods of the first aspect and suitable methods of ameliorating metabolic dysfunction include methods of the second aspect.

The heterologous nucleic acid of the first to the fourth aspects may be contained in a recombinant viral vector or viral particle, such as a recombinant adenovirus associated viral (AAV) vector or viral particle.

The lipodystrophy of the first to the fourth aspects may be a congenital generalised lipodystrophy (CGL). The CGL may be characterised by an autosomal recessive defective gene. The heterologous nucleic acid may be encoded a therapeutic protein i.e. a functional version of the protein that is encoded by the defective gene that characterises the CGL.

In some embodiments, the CGL is congenital generalised lipodystrophy type 2 (CGL type 2). CGL type 2 is characterised by a defective BSCL2 gene. A method of treatment of CGL type 2 may comprise administering to an individual in need thereof a heterologous nucleic acid encoding BSCL2 (also known as seipin).

In other embodiments, the congenital lipodystrophy is congenital generalised lipodystrophy type 1 (CGL type 1). CGL type 1 is characterised by a defective AGPAT2 gene. A method of treatment of CGL type 1 may comprise administering to an individual in need thereof a heterologous nucleic acid encoding AGPAT2.

In other embodiments, the congenital lipodystrophy is congenital generalised lipodystrophy type 3 (CGL type 3). CGL type 3 is characterised by a defective CAV1 gene. A method of treatment of CGL type 3 may comprise administering to an individual in need thereof a heterologous nucleic acid encoding CAV1.

In other embodiments, the congenital lipodystrophy is congenital generalised lipodystrophy type 4 (CGL type 4). CGL type 4 is characterised by a defective CAVIN1 gene. A method of treatment of CGL type 4 may comprise administering to an individual in need thereof a heterologous nucleic acid encoding CAVIN1.

The lipodystrophy of the first to the fourth aspects may be a familial partial lipodystrophy (FPLD).

In some embodiments, the FPLD may be characterised by an autosomal recessive defective gene. The heterologous nucleic acid may encode a therapeutic protein i.e. a functional version of the protein that is encoded by the defective recessive gene. Defective recessive genes that may characterise FPLD may include CIDEC. For example, the FLPD may be characterised by a defective CIDEC gene. A method of treatment of FPLD may comprise administering to an individual in need thereof a heterologous nucleic acid encoding a functional version of the protein encoded by the defective recessive gene. For example, a method of treatment of FPLD may comprise administering to an individual in need thereof a heterologous nucleic acid encoding CIDEC.

In other embodiments, the FPLD may be characterised by an autosomal dominant defective gene. The heterologous nucleic acid may encode an RNA molecule that inhibits expression from the defective dominant gene. Defective dominant genes that may characterise FPLD may include AKT2, LMNA, PPARG, ZMPSTE24, PLIN1, MFN2, PCTY1A, FBN1, POLD1, PIK3R1, and PSMB8 A method of treatment of FPLD may comprise administering to an individual in need thereof a heterologous nucleic acid encoding an RNA molecule that inhibits expression from the defective dominant gene. For example, the method may comprise administering to an individual in need thereof a heterologous nucleic acid encoding an RNA molecule that inhibits expression from the defective AKT2, LMNA, PPARG, ZMPSTE24, PLIN1, MFN2, PCTY1A, FBN1, POLD1, PIK3R1, or PSMB8 gene.

Other aspects and embodiments of the invention are described in more detail below.

### BRIEF DESCRIPTION OF THE FIGURES

FIG. 1 shows adeno-associated virus (AAV) delivery of eGFP to murine tissues. (A) Western blot analysis of eGFP levels in liver, gonadal white adipose tissue (gWAT), subcutaneous white adipose tissue (sWAT) and brown adipose tissue (BAT). Male mice were injected with AAV8 vectors overexpressing eGFP from the mammalian cytomegalovirus (CMV) promoter. Intraperitoneal (I.P.) or intravenous (I.V.) injections were made using 1×1010, 1×1011 or 1×1012 genome copies of AAV or an equivalent volume of PBS, which was used as a control. (B) Immunohistochemistry of eGFP in liver, gWAT, sWAT and BAT sections in mice receiving 1×1012 genome copies of AAV or an equivalent volume of PBS by I.P. Scale bar represents 20 μm. (C) Viral vector design to overexpress the long form of the human BSCL2 transcript driven by the CMV promoter (AAV-CMV-hBSCL2). (D) Graphical representation of the experimental strategy used to determine whether gene therapy can rescue metabolic dysfunction in seipin knockout mice (SKO).

FIG. 2 shows that gene therapy prevents weight gain and rescues hyperglycaemia in seipin knockout mice. (A) Weight gain progression in wild type (WT) and SKO mice after I.P. injection of 1×10¹² genome copies of AAV vectors containing eGFP (SKO-eGFP) or hBSCL2 (SKO-hBSCL2). Serum glucose levels in ad lib fed WT, SKO-eGFP and SKO-hBSCL2 mice at two (B) and nineteen (C) weeks after administration of gene therapy. Whole body fat mass (D) and whole-body lean mass (E) levels assessed by Echo-MRI prior to gene therapy (Pre-AAV), two and nineteen weeks after AAV administration. (F) Percentage of lean mass gained nineteen weeks after AAV administration compared to Pre-AAV lean mass values. All data are biological replicates presented as the mean±SEM, n=8-12 mice per group, * p<0.05, ** p<0.01, *** p<0.001 and **** p<0.0001 vs WT, #p<0.05, ##p<0.01 and ###p<0.001 vs SKO-eGFP.

FIG. 3 shows that gene therapy rescues hepatomegaly and restores adipose tissue development in seipin knockout mice. Tissue weight, dissection images and H&E histology sections of liver (A) and gWAT (D) from WT, SKO-eGFP and SKO-hBSCL2 male mice twenty weeks after AAV administration. Scale bar represents 80 μm. Western blot analysis of eGFP and hBSCL2 protein levels in liver (B) and hBSCL2 protein levels in gWAT (E) in WT, SKO-eGFP and SKO-hBSCL2 mice twenty weeks after AAV administration. One male and one female are presented in each condition. Relative gene expression levels of metabolic markers in the liver (C) and markers of adipogenesis in gWAT (F) from WT, SKO-eGFP and SKO-hBSCL2 mice twenty weeks after AAV administration. All data are biological replicates presented as the mean±SEM, n=3-6 mice per group for A and D, n=8-11 mice for C and F, *p<0.05, ** p<0.01, *** p<0.001 and **** p<0.0001 vs WT, ##p<0.01 and ###p<0.001 vs SKO-eGFP, ntd=no tissue dissected, nd=not detected.

FIG. 4 shows that seipin knockout mice administered gene therapy are insulin sensitive. Liver (A) and serum (B) triglyceride (TG) levels measured in WT, SKO-eGFP and SKO-hBSCL2 mice fasted for five hours and twenty weeks after AAV administration. Relative gene expression (C) and circulating serum levels (D) of leptin and adiponectin in WT, SKO-eGFP and SKO-hBSCL2 mice fasted for five hours. Serum insulin (E), glucose (F) and quantitative insulin sensitivity check index (QUICKI) analysis (G) in WT, SKO-eGFP and SKO-hBSCL2 mice fasted for five hours. All data are biological replicates presented as the mean±SEM, n=8-11 mice per group, * p<0.05, ** p<0.01 and *** p<0.001 vs WT and #p<0.05, ##p<0.01 and ###p<0.001 vs SKO-eGFP by one-way ANOVA, xxx p<0.001 and xxxx p<0.0001 vs SKO-eGFP by unpaired t-test, ntd=no tissue dissected.

FIG. 5 shows adeno-associated virus (AAV) delivery of eGFP to murine tissues. (A) Western blot analysis of eGFP levels in muscle, heart, kidney and testis. Male mice were injected with AAV8 vectors overexpressing eGFP from the mammalian cytomegalovirus (CMV) promoter. Intraperitoneal (I.P.) or intravenous (I.V.) injections were made using 1×10¹⁰, 1×10¹¹ or 1×10¹² genome copies of AAV or an equivalent volume of PBS, which was used as a control. (B) Immunohistochemistry of eGFP in liver, gWAT, sWAT and BAT sections in mice receiving 1×10¹² genome copies of AAV or an equivalent volume of PBS by I.V. Scale bar represents 20 μm.

FIG. 6 shows the characterisation of seipin knockout mice treated with gene therapy. Whole body fat mass (A) and whole-body lean mass (B) levels assessed by Echo-MRI and normalised to body weight prior to gene therapy (Pre-AAV), at two and nineteen weeks after AAV administration in WT, SKO-eGFP and SKO-hBSCL2 mice. Relative gene expression levels of eGFP and hBSCL2 in the liver (C). Tissue weight of (D) retroperitoneal white adipose tissue (rWAT) and (E) brown adipose tissue (BAT) from WT, SKO-eGFP and SKO-hBSCL2 male mice twenty weeks after AAV administration. Relative gene expression levels of hBSCL2 in gWAT (F). Homeostatic model assessment of insulin resistance (HOMA-IR) analysis of WT, SKO-eGFP and SKO-hBSCL2 mice twenty weeks after AAV administration. All data are biological replicates presented as the mean±SEM, n=8-12 mice per group for A, B, C, F and G, n=3-6 mice per group for D and E, * p<0.05, ** p<0.01 and *** p<0.001 vs WT, ##p<0.01 vs SKO-eGFP, ntd=no tissue dissected, nd=not detected.

### DETAILED DESCRIPTION

This invention relates to the treatment of lipodystrophy and/or the amelioration of metabolic dysfunction associated with lipodystrophy by the administration of a recombinant viral particle, for example a recombinant AAV particle. The recombinant viral particle may comprise a heterologous nucleic acid that encodes the functional product of a gene whose defectiveness characterises the lipodystrophy. Expression of the gene product encoded by the heterologous nucleic acid may, for example, restore or generate adipose tissue in the patient, thereby treating the lipodystrophy and/or ameliorating metabolic dysfunction in the patient.

Lipodystrophy is characterised by the absence of adipose tissue, for example metabolic adipose tissue, such as subcutaneous or visceral adipose tissue, and/or mechanical adipose tissue, such as plantar or retro-orbital adipose tissue. Individuals with lipodystrophy may be unable to produce adipose tissue or may be unable to maintain adipose tissue. Individuals with lipodystrophy may lack functional adipocytes. The absence of adipose tissue impairs the ability of the individual to store lipids, resulting in the accumulation of lipids in ectopic sites. This leads to metabolic dysfunction, including insulin resistance.

Lipodystrophy may be partial or generalised. A generalised lipodystrophy, such as a CGL, may be characterised by the total or near total absence of adipose tissue in the individual. A partial lipodystrophy, such as a FPLD, may be characterised by the abnormal distribution of adipose tissue. For example, adipose tissue may be depleted or absent from certain parts of the individual.

The lipodystrophy may be a congenital generalised lipodystrophy. Congenital generalised lipodystrophy is a heritable condition that is characterised by the absence of adipose tissue from birth. A congenital generalised lipodystrophy may be caused by a defective gene i.e. a lipodystrophy gene that contains a genetic defect. Congenital lipodystrophies may include congenital generalised lipodystrophy type 2 (CGL type 2); congenital generalised lipodystrophy type 1 (CGL type 1); congenital generalised lipodystrophy type 3 (CGL type 3); and congenital generalised lipodystrophy type 4 (CGL type 4).

The lipodystrophy may be a familial partial lipodystrophy (FPLD). FPLD is a heritable condition that is characterised by the selective loss or absence of adipose tissue, for example subcutaneous fat, from certain parts of the body. A FPLD may be caused by a defective gene i.e. a lipodystrophy gene that contains a genetic defect. FPLDs may include FPLD type 2; FPLD type 3; FPLD type 4; FPLD type 5; and FPLD type 6.

Lipodystrophy suitable for treatment as described herein may arise from a defective gene. A gene which, when defective, causes or is associated with lipodystrophy may be referred to herein as a lipodystrophy gene. A defective lipodystrophy gene may be a characteristic feature of a lipodystrophy. For example, a lipodystrophy may be characterised by a genetic mutation, such as an insertion, deletion, or substitution, in a lipodystrophy gene.

A defective lipodystrophy gene may encode an inactive gene product or a gene product with aberrant activity or may not express an active gene product. For example, the defective gene may contain genetic mutation that reduces or abolishes the expression of the gene or reduces or abrogates the activity of the protein or other gene product encoded by the gene. Genetic mutations may include nonsense mutations, missense mutations, spice-site variants, insertions and deletions and may lead to the expression of an inactive gene product.

Lipodystrophy genes may be autosomal recessive genes or autosomal dominant gene. An autosomal recessive lipodystrophy gene may give rise to lipodystrophy in an individual when both copies of the lipodystrophy gene are defective. Expression of a heterologous nucleic acid encoding a functional version of a protein encoded by a defective autosomal recessive lipodystrophy gene, as described herein, may ameliorate or treat the condition. An autosomal dominant lipodystrophy gene may give rise to lipodystrophy in an individual when a single copy of the lipodystrophy gene is defective. Expression of a heterologous nucleic acid encoding an RNA that inhibits expression from a protein encoded by a defective autosomal dominant lipodystrophy gene, as described herein, may ameliorate or treat the condition.

Lipodystrophy genes associated with CGL may include the autosomal recessive genes BSCL2 (CGL2), AGPAT2 (CGL1), CAV1 (CGL3), and CAVIN1 (CGL4).

Defects in Berardinelli-Seip Congenital Lipodystrophy 2 (BSCL2) may cause congenital generalised lipodystrophy type 2 (CGL type 2). BSCL2 (Gene ID: 26580, also known as HMN5; PELD; SPG17; GNG3LG) encodes seipin, which is a transmembrane protein that has been found to induce lipid droplet fusion. Human seipin isoform 1 may have the reference amino acid sequence of NP_001116427.1 or SEQ ID NO: 2 and may be encoded by the reference nucleic acid sequence of NM_00122955.4 or SEQ ID NO: 1. Reference amino acid and nucleotide sequences for other seipin isoforms are available on public sequence databases (e.g. NCBI, Bethesda MD USA).

Defects in acylglycerol-3-phosphate-O-acyltransferase 2 (AGPAT2) may cause congenital generalised lipodystrophy type 1 (CGL type 1). AGPAT2 (Gene ID: 10555, also known as BSCL; BSCL1; LPAAB; 1-AGPAT2; LPAAT-beta) encodes an ER protein that converts lysophosphatidic acid to phosphatidic acid and plays a critical role in the synthesis of glycerophospholipids and triglycerides required for lipid droplet formation. Human AGPAT2 isoform 2 may have the reference amino acid sequence of NP_001012745.1 or SEQ ID NO: 4 and may be encoded by the reference nucleic acid sequence of NM_001012727.2 or SEQ ID NO: 3. Reference amino acid and nucleotide sequences for other AGPAT2 isoforms are available on public sequence databases (e.g. NCBI, Bethesda MD USA). AGPAT2 activity may be determined by standard techniques in the art (see for example, Haque et al (2005) Biochem Biophys Res Commun 2005; 327: 446-453).

Defects in caveolin 1 (CAV1) may cause congenital generalised lipodystrophy type 3 (CGL type 3). CAV1 (Gene ID 857; CGL3; also known as PPH3; BSCL3; LCCNS; VIP21; MSTP085) is an integral component of caveolae plasma membranes and may contribute towards lipid droplet formation. Human caveolin 1 variant 2 may have the reference amino acid sequence of NP_001166366.1 or SEQ ID NO: 6 and may be encoded by the reference nucleic acid sequence of NM_001172895.1 or SEQ ID NO: 5. Reference amino acid and nucleotide sequences for other CAV1 variants are available on public sequence databases (e.g. NCBI, Bethesda MD USA)

Defects in caveolae associated protein 1 (CAVIN1) may cause congenital generalised lipodystrophy type 4 (CGL type 4). CAVIN1 (Gene ID: 284119; also known as CGL4; PTRF; CAVIN; FKSG13; cavin-1) promotes the dissociation of transcription complexes and plays a key role in the formation of caveolae and the stabilization of caveolin. Human CAVIN1 may have the reference amino acid sequence of NP_036364.2 and may be encoded by the reference nucleic acid sequence of NM_012232.6. Reference amino acid and nucleotide sequences for other CAVIN1 isoforms are available on public sequence databases (e.g. NCBI, Bethesda MD USA).

Defects in BSCL2 (CGL2), AGPAT2 (CGL1), CAV1 (CGL3), and CAVIN1 (CGL4) that cause congenital lipodystrophy are well-established in the art (see for example Craviero Sarmento et al (2019) Mutat Res Rev Mutat Res 781 30-52).

Lipodystrophy genes associated with FPLD may include the autosomal recessive genes CIDEC (FPLD5) and LIPE (FPLD6) (see for example Mann et al (2019) J Clin Invest 129(10) 4009-4021).

Defects in cell death inducing DFFA like effector c (CIDEC) may cause familial partial lipodystrophy type 4 (FPLD5). CIDEC (Gene ID: 63924; also known as CIDE3; FPLD5; FSP27; CIDE-3) promotes lipid droplet formation in adipocytes and mediates adipocyte apoptosis. Human CIDEC may have the reference amino acid sequence of NP_001186480.1 and may be encoded by the reference nucleic acid sequence of NM_001199551.2. Reference amino acid and nucleotide sequences for other CIDEC isoforms are available on public sequence databases (e.g. NCBI, Bethesda MD USA).

Defects in lipase E (LIPE) may cause familial partial lipodystrophy type 6 (FPLD6). LIPE (Gene ID: 3991; also known as HSL; LHS; REH; AOMS4; FPLD6) converts cholesteryl esters to free cholesterol and hydrolyzes stored triglycerides to free fatty acids. Human LIPE may have the reference amino acid sequence of NP_005348.2 and may be encoded by the reference nucleic acid sequence of NM_005357.4. Reference amino acid and nucleotide sequences for other LIPE isoforms are available on public sequence databases (e.g. NCBI, Bethesda MD USA).

Lipodystrophy genes associated with FPLD may include the autosomal dominant genes LMNA (FPLD2), PPARG (FPLD3), PLIN1 (FPLD4), AKT2 and ADRA2A (see for example Mann et al (2019) J Clin Invest 129(10) 4009-4021).

Lamin A/C (LMNA) may cause familial partial lipodystrophy type 2 (FPLD2). LMNA (Gene ID: 4000; also known as FPLD2; LMNL1; CMT2B1; LGMD1B) is part of the matrix of proteins located next to the inner nuclear membrane and plays roles in nuclear stability, chromatin structure and gene expression. Human LMNA may have the reference amino acid sequence of NP_001244303.1 and may be encoded by the reference nucleic acid sequence of NM_001257374.3. Reference amino acid and nucleotide sequences for other LMNA isoforms are available on public sequence databases (e.g. NCBI, Bethesda MD USA).

Peroxisome proliferator activated receptor gamma (PPARG) may cause familial partial lipodystrophy type 3 (FPLD3). PPARG (Gene ID: 5468; also known as GLM1; CIMT1; NR1C3; PPARG1) form heterodimers with retinoid X receptors (RXRs) to regulate transcription of various genes. Human PPARG may have the reference amino acid sequence of NP_001317544.2 and may be encoded by the reference nucleic acid sequence of NM_001330615.4. Reference amino acid and nucleotide sequences for other PPARG isoforms are available on public sequence databases (e.g. NCBI, Bethesda MD USA).

Perilipin 1 (PLIN1) may cause familial partial lipodystrophy type 4 (FPLD4). PLIN1 (Gene ID: 5346; also known as PERI; PLIN; FPLD4) coats lipid storage droplets in adipocytes and inhibits lipolysis. Human PLIN1 may have the reference amino acid sequence of NP_001138783.1 and may be encoded by the reference nucleic acid sequence of NM_001145311.2. Reference amino acid and nucleotide sequences for other PLIN1 isoforms are available on public sequence databases (e.g. NCBI, Bethesda MD USA).

AKT serine/threonine kinase 2 (AKT2) may cause AKT-related familial partial lipodystrophy. AKT2 (Gene ID: 208; also known as PKBB; PRKBB; HIHGHH) is a kinase within the insulin signalling cascade that is required for in vitro adipogenesis. Human AKT2 may have the reference amino acid sequence of NP_001229956.1 and may be encoded by the reference nucleic acid sequence of NM_001243027.3. Reference amino acid and nucleotide sequences for other AKT2 isoforms are available on public sequence databases (e.g. NCBI, Bethesda MD USA).

Adrenoceptor alpha 2A (ADRA2A) may cause atypical familial partial lipodystrophy. ADRA2A (Gene ID: 150; also known as ADRA2; ADRAR; ZNF32) is involved in presynaptic transmitter release from the sympathetic nervous system in the heart and from central noradrenergic neurons. Human ADRA2A may have the reference amino acid sequence of NP_00672.3 and may be encoded by the reference nucleic acid sequence of NM_00681.4. Reference amino acid and nucleotide sequences for other ADRA2A isoforms are available on public sequence databases (e.g. NCBI, Bethesda MD USA).

Lipodystrophy genes associated with other lipodystrophies include the autosomal recessive genes MFN2, PCTY1A, ZMPSTE24, and PSMB8 and the autosomal dominant genes FBN1, POLD1 and PIK3R1.

Mitofusin 2 (MFN2) may cause atypical familial partial lipodystrophy. MFN2 (Gene ID: 9927; also known as HSG; MARF; CMT2A; CPRP1; CMT2A2) is a mitochondrial membrane protein that regulates vascular smooth muscle cell proliferation. Human MFN2 may have the reference amino acid sequence of NP_001121132.1 and may be encoded by the reference nucleic acid sequence of NM_001127660.2. Reference amino acid and nucleotide sequences for other MFN2 isoforms are available on public sequence databases (e.g. NCBI, Bethesda MD USA).

Phosphate cytidylyltransferase 1, choline, alpha (PCTY1A) may cause atypical congenital lipodystrophy. PCTY1A (Gene ID: 5130; also known as CT; CTA; CCTA; CTPCT; PCYT1; SMDCRD) regulates phosphatidylcholine biosynthesis. Human PCTY1A may have the reference amino acid sequence of NP_001299602.1 and may be encoded by the reference nucleic acid sequence of NM_001312673.2. Reference amino acid and nucleotide sequences for other PCTY1A isoforms are available on public sequence databases (e.g. NCBI, Bethesda MD USA).

Zinc metallopeptidase STE24 (ZMPSTE24) may cause atypical familial partial lipodystrophy. ZMPSTE24 (Gene ID: 10269; also known as HGPS; PRO1; FACE1; STE24; FACE-1; Ste24p) cleaves carboxy terminal residues of farnesylated prelamin A to form mature lamin A. Human ZMPSTE24 may have the reference amino acid sequence of NP_005848.2 and may be encoded by the reference nucleic acid sequence of NM_005857.5. Reference amino acid and nucleotide sequences for other ZMPSTE24 isoforms are available on public sequence databases (e.g. NCBI, Bethesda MD USA).

Proteasome 20S subunit beta 8 (PSMB8) may cause atypical familial partial lipodystrophy. PSMB8 (Gene ID: 5696; also known as JMP; ALDD; LMP7; NKJO; D6S216) is a proteasome component whose expression is induced by IFNγ. Human PSMB8 may have the reference amino acid sequence of NP_004150.1 and may be encoded by the reference nucleic acid sequence of NM_004159.5. Reference amino acid and nucleotide sequences for other PSMB8 isoforms are available on public sequence databases (e.g. NCBI, Bethesda MD USA).

Fibrillin 1 (FBN1) may cause congenital lipodystrophy. FBN1 (Gene ID: 2200; also known as FBN; SGS; WMS; MASS) is an extracellular matrix glycoprotein that serves as a structural component of calcium-binding microfibrils. Human FBN1 may have the reference amino acid sequence of NP_00129.3 and may be encoded by the reference nucleic acid sequence of NM_00138.5. Reference amino acid and nucleotide sequences for other FBN1 isoforms are available on public sequence databases (e.g. NCBI, Bethesda MD USA).

DNA polymerase delta 1, (POLD1) may cause congenital lipodystrophy. POLD1 (Gene ID: 5424; also known as CDC2; MDPL; POLD; CRCS10) is involved in DNA replication and repair. Human POLD1 may have the reference amino acid sequence of NP_001243778.1 and may be encoded by the reference nucleic acid sequence of NM_001256849.1. Reference amino acid and nucleotide sequences for other POLD1 isoforms are available on public sequence databases (e.g. NCBI, Bethesda MD USA).

Phosphoinositide-3-kinase regulatory subunit 1 (PIK3R1) may cause atypical familial partial lipodystrophy. PIK3R1 (Gene ID: 5295; also known as p85; AGM7; GRB1; IMD36) phosphorylates the inositol ring of phosphatidylinositol. Human PIK3R1 may have the reference amino acid sequence of NP_001229395.1 and may be encoded by the reference nucleic acid sequence of NM_001242466.2. Reference amino acid and nucleotide sequences for other PIK3R1 isoforms are available on public sequence databases (e.g. NCBI, Bethesda MD USA).

In the methods described herein, a heterologous nucleic acid encodes a functional version of the protein that is encoded by an autosomal recessive gene that is defective in the lipodystrophy or an RNA molecule that inhibits expression from an autosomal dominant gene that is defective in the lipodystrophy. The functional version encoded by the heterologous nucleic acid may be referred to herein as a “therapeutic polypeptide”. The RNA molecule encoded by the heterologous nucleic acid may be referred to herein as a “therapeutic RNA molecule”. Expression of the gene product encoded by the heterologous nucleic acid may generate adipose tissue in the individual and may alleviate or reduce symptoms of lipodystrophy in an individual or may otherwise confer a benefit to the individual, for example by improving metabolic function or reducing metabolic dysfunction. Preferably, the gene product is expressed by the heterologous nucleic acid in adipocyte precursor stem cells of immature pre-adipocytes. Expression of the gene product may convert these cells in functional adipocytes.

The expression of a therapeutic gene product, such as a protein or RNA molecule, in a cell or tissue may be determined by standard techniques.

The term “heterologous” refers to a polypeptide or nucleic acid that is foreign to a particular biological system, such as a host cell, and is not naturally occurring in that system. A heterologous polypeptide or nucleic acid may be introduced to a biological system by artificial means, for example using recombinant techniques. For example, a heterologous nucleic acid may be a nucleic acid or nucleotide sequence that does not naturally occur in the adenovirus associated virus i.e. the heterologous nucleic acid has been artificially introduced into the AAV expression vector by recombinant means. A heterologous nucleic acid encoding a therapeutic polypeptide or RNA molecule may be inserted into a suitable expression construct which is in turn used to transform a host cell to produce the polypeptide or RNA molecule. A heterologous polypeptide or nucleic acid may be synthetic or artificial or may exist in a different biological system, such as a different species or cell type.

The protein encoded by the heterologous nucleic acid may comprise the non-mutated amino acid sequence of the protein that is encoded by the gene that is defective in the lipodystrophy, or may be a variant thereof. In some embodiments, the heterologous nucleic acid may comprise the wild-type non-mutated coding sequence of the gene that is defective in the lipodystrophy, or a variant thereof.

A variant of a reference sequence referenced or set out herein may comprise an amino acid sequence or a nucleotide sequence having at least 50%, at least 60%, at least 70%, at least 80%, at least 90%, at least 95%, or at least 98% sequence identity to the reference sequence. Particular amino acid sequence variants may differ from the reference sequence by insertion, addition, substitution or deletion of 1 amino acid, 2, 3, 4, 5, 6, 7, 8, 9, or 10 or more than 10 amino acids. Particular nucleotide sequence variants may differ from the reference sequence by insertion, addition, substitution or deletion of 1 nucleotide, 2, 3, 4, 5, 6, 7, 8, 9, or 10 or more than 10 nucleotides.

Sequence similarity and identity are commonly defined with reference to the algorithm GAP (Wisconsin Package, Accelerys, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used. Computerized implementations of these algorithms (GAP, BESTFIT, PASTA, and FASTA in the Wisconsin Genetics Software Package, Genetics Computer Group, 575 Science Dr., Madison, WI) are available and publicly available computer software may be used such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)), Genomequest™ software (Gene-IT, Worcester MA USA) and MAFFT (Katoh and Standley 2013, Molecular Biology and Evolution, 30(4) 772-780 software. When using such software, the default parameters, e.g. for gap penalty and extension penalty, are preferably used. A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Sequence comparison may be made over the full-length of the relevant sequence described herein.

A variant of a reference sequence may have the same function or activity as the reference sequence.

The RNA molecule encoded by the heterologous nucleic acid may suppress expression of a defective lipodystrophy gene with autosomal dominance. The RNA molecule suppresses expression of the defective copy of the lipodystrophy gene, such that only expression of the non-defective copy of the lipodystrophy gene remains. This may ameliorate or treat lipodystrophy or metabolic dysfunction associated with lipodystrophy, as described herein.

Suitable RNA molecules include shRNA molecules and miRNA molecules.

RNAi involves the expression or introduction into a cell of an RNA molecule which comprises a sequence which is identical or highly similar to the coding sequence of the defective copy of the lipodystrophy gene. The RNA molecule interacts with mRNA which is transcribed from the defective copy of the lipodystrophy gene, resulting in the sequence specific degradation or specific post-transcriptional gene silencing (PTGS) of the mRNA. This reduces or suppresses expression of active protein from the defective copy of the lipodystrophy gene (Angell & Baulcombe (1997) The EMBO Journal 16, 12:3675-3684; Voinnet & Baulcombe (1997) Nature 389: pg 553).

The RNA molecule is preferably double stranded RNA (dsRNA) (Fire A. et al Nature 391, (1998)). Synthetic siRNA duplexes have been shown to specifically suppress expression of endogenous and heterologous genes in a wide range of mammalian cell lines (Elbashir SM. et al. Nature, 411, 494-498, (2001)).

Suitable RNA molecules for use in RNAi suppression include short interfering RNA (siRNA). siRNA are double stranded RNA molecules of 15 to 40 nucleotides in length, preferably 15 to 28 nucleotides or 19 to 25 nucleotides in length, for example 19, 20, 21, 22, 23, 24 or 25 nucleotides in length. For example, two unmodified 21 mer oligonucleotides may be annealed together to form a siRNA. A siRNA molecule may contain a 3′ and/or 5′ overhang on each strand having a length of about 0, 1, 2, 3, 4, or 5 nucleotides. The overhang lengths of the strands are independent, i.e., the length of the overhang on one strand is not dependent on the length of the overhang on the second strand.

Other suitable RNA molecules for use in RNAi include small hairpin RNAs (shRNAs). shRNA are single-chain RNA molecules which consist of short inverted repeats separated by a small loop sequence. A shRNA may comprise or consist of a short (e.g. 19 to 25 nucleotides) antisense nucleotide sequence, followed by a nucleotide loop of 5 to 9 nucleotides, and the complementary sense nucleotide sequence (e.g. 19 to 25 nucleotides). Alternatively, the sense sequence may precede the nucleotide loop structure and the antisense sequence may follow. The nucleotide loop forms a hairpin turn which allows the base pairing of the complementary sense and antisense sequences to form the shRNA.

The complementary sense or antisense nucleotide sequence (or inverted repeat) may be complementary to the gene target.

In the cell the shRNA is processed by DICER into a siRNA which degrades the target gene mRNA and suppresses expression. In a preferred embodiment the shRNA is produced endogenously (within a cell) by transcription from a viral vector. shRNAs may be produced within a cell by transfecting the cell with a vector encoding the shRNA sequence under control of a RNA polymerase III promoter such as the human H1 or 7SK promoter or a RNA polymerase II promoter. For example, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure. Lentiviral vectors, adenoviral vectors and other viral vectors have been successfully utilised in various gene delivery applications including the delivery of small hairpin RNA (shRNA) for RNA interference (RNAi) (Moffat, J. et al. Cell 124, 1283-1298 (2006); Silva, J. M. et al. Nature genetics 37, 1281-1288 (2005)

An RNA molecule, such as an siRNA or shRNA, may comprise or consist of a sequence which is identical or substantially identical (i.e. at least 90%, at least 95% or at least 98% identical) to all or part (for example, 15 to 40 nucleotides) of a reference nucleotide sequence of a defective copy of a lipodystrophy gene, or its complement.

RNA molecules, such as siRNAs and shRNAs, for reducing expression of a defective copy of a lipodystrophy gene may be readily designed using reference nucleotide sequences and software tools which are widely available in the art and may be produced using routine techniques. For example, a suppressor nucleic acid may be chemically synthesized; produced recombinantly in vitro or cells (Elbashir, S. M. et al., Nature 411:494-498 (2001); Elbashir, S. M., et al., Genes & Development 15:188-200 (2001)) or obtained from commercial sources (e.g. Cruachem (Glasgow, UK), Dharmacon Research (Lafayette, Colo., USA)).

The heterologous nucleic acid that encodes the RNA molecule that inhibits expression of the defective gene or the functional version of the product of the gene that is defective in the lipodystrophy may be operably linked to one or more control elements or regulatory sequences capable of directing the in vivo expression of the therapeutic protein. Preferably the one or more control elements or regulatory sequences are heterologous i.e. they do not naturally occur in operable linkage to the lipodystrophy-associated gene.

Suitable control elements or regulatory sequences to drive the expression of heterologous nucleic acid coding sequences in mammalian cells, preferably human cells, are well-known in the art and include constitutive promoters, for example viral promoters such as CMV or SV40; and tissue specific promoters, for example liver specific promoters, such as the TBG promoter; or adipose specific promoters, such as the mini/aP2 promoter or adiponectin (APM1) promoter.

The heterologous nucleic acid may be delivered to the individual in the methods described herein in a recombinant viral vector or viral particle. Suitable viral vectors include retroviral vectors, such as lentiviral vectors, herpes simplex viral (HSV) vectors and adenoviral vectors and adenovirus associated viral (AAV) vectors.

In some preferred embodiments, the recombinant viral vector or viral particle is a recombinant adenovirus associated viral (AAV) vector or viral particle.

Adeno-associated virus (AAV) is a DNA virus of relatively small size (about 20 nm diameter) that can integrate, in a stable and site-specific manner, into the genome of the cells that they infect. AAV is able to infect a wide spectrum of cells without inducing significant effects on cellular growth, morphology or differentiation. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV of any serotype are suitable for use as expression vectors as described herein (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” in Parvoviruses (J R Kerr, S F Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) 15-23, Hudder Arnold, London, UK (2006)). Suitable AAV expression vectors as described herein include those encapsidated into a virus particle (e.g. AAV virus particle) including, AAV1, AAV2, AAV3, AAV4, AAV5, AAV6, AAV7, AAV8, AAV9, AAV10, AAV11, AAV12, AAV13, AAV14, AAV15, and AAV16.

In some preferred embodiments, the AAV may be AAV6, AAV8 or AAV9, preferably AAV8.

Recombinant adeno-associated viruses (rAAVs) have been used extensively as expression vectors for transferring genes in vitro and in vivo. In some embodiments, a rAAV vector may be generated in which the rep and/or cap genes of the AAV virus are deleted and replaced by a heterologous nucleic acid. A suitable rAAV vector may comprise a viral genome that includes at least one inverted terminal repeat (e.g., one, two or three inverted terminal repeats) and one or more heterologous nucleic acid sequences. rAAV vectors generally retain the 145 base inverted terminal repeat(s) (ITR(s)) in cis to generate virus; however, modified AAV ITRs and non-AAV ITRs, including partially or completely synthetic sequences, can also serve this purpose. All other viral sequences are dispensable and may be supplied in trans (see for example Muzyczka, (1992) Curr. Topics Microbiol. Immunol. 158:97). A suitable rAAV vector for use in delivering heterologous nucleic acid as described herein may comprise two ITRs (e.g. AAV ITRs), which generally will be at the 5′ and 3′ ends of the heterologous nucleic acid but need not be contiguous thereto. The ITRs can be the same or different from each other. The use of rAAV vectors for gene therapy is well established in the art (see for example Naso et al BioDrugs. 2017; 31(4): 317-334; Daya et al Clin Microbiol Rev. 2008 October; 21(4): 583-593).

Recombinant AAV vectors may be produced and then purified using techniques that are standard in the art (see for example, US65661 18, U.S. Pat. Nos. 6,989,264, 6,995,006 and WO1999/011764). For example, AAV viral particles may be produced in vitro by a method involving transducing mammalian cells with a viral vector or expression vector as described herein and expressing viral packaging and envelope proteins necessary for particle formation in the cells and culturing the transduced cells in a culture medium, such that the cells produce viral particles that are released into the medium.

An AAV particle may comprise an AAV expression vector comprising the heterologous nucleic acid encapsidated in a viral capsid, preferably an AAV viral capsid.

It is possible to use a single expression vector that encodes all the viral components required for viral particle formation and function. Most often, however, multiple plasmid expression vectors or individual expression cassettes integrated stably into a host cell, such as a human embryonic kidney (HEK) 293 cell, are utilised to separate the various genetic components that generate the viral vector particles.

In some embodiments, expression cassettes encoding the one or more viral packaging and envelope proteins have been integrated stably into a mammalian cell. In these embodiments, transducing these cells with a viral vector described herein is sufficient to result in the production of viral particles without the addition of further expression vectors.

In other embodiments, the in vitro methods may involve using multiple expression vectors. In some embodiments, the method comprises transducing the mammalian cells with one or more expression vectors encoding the viral packaging and envelope proteins that encode the viral packaging and envelope proteins necessary for particle formation. For example, a recombinant AAV vector may be prepared by co-transfecting a plasmid containing the heterologous nucleic acid flanked by two AAV inverted terminal repeat (ITR) regions, and a plasmid carrying the AAV encapsidation genes (rep and cap genes), into a cell line that is infected with a human helper virus (for example an adenovirus).

Suitable methods for producing rAAV particles are well-known in the art.

Following release of viral particles, the culture medium comprising the rAAV particles may be collected and, optionally the viral particles may be separated from the culture medium. Optionally, the viral particles may be concentrated and/or purified. Suitable techniques for the manufacture of AAV expression vectors are well established in the art (Clement N, Grieger J C. Mol Ther Methods Clin Dev. 2016 Mar. 16: 3: 16002).

Following production and optional concentration, the viral particles may be stored, for example by freezing at −80° C. ready for use in therapy as described herein.

While it is possible for a recombinant viral particle, such as a recombinant AAV particle, to be used (e.g., administered) alone, it is often preferable to present it as a composition or formulation e.g. with a pharmaceutically acceptable excipient, carrier or diluent.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation. Suitable carriers, excipients, etc. can be found in standard pharmaceutical texts, for example, Remington's Pharmaceutical Sciences, 18th edition, Mack Publishing Company, Easton, Pa., 1990. Suitable pharmaceutically acceptable excipients include phosphate-buffered saline (PBS).

A pharmaceutical composition (e.g., formulation, preparation, medicament) comprising, or consisting essentially of, or consisting of as a sole active ingredient, a viral particle, such as a rAAV particle, may conveniently be presented in unit dosage form. The unit dose may be calculated in terms of the dose of viral particles being administered. Viral doses include a particular number of virus particles or plaque forming units (pfu). For embodiments involving adenovirus, particular unit doses include 10³, 10⁴, 10⁵, 10⁶, 10⁷, 10⁸, 10⁹, 10¹⁰, 10¹¹, 10¹², 10¹³ or 10¹⁴ pfu. Particle doses may be somewhat higher (10 to 100 fold) due to the presence of infection-defective particles. In some embodiments, 10¹² to 10¹⁴ genome copies of the recombinant viral particle may be administered.

The viral particle or pharmaceutical composition comprising the viral particle may be administered to an individual as treatment for lipodystrophy.

An individual suitable for treatment may totally or partially lack adipose tissue. For example, the individual may totally or partially lack adipocytes. The individual may have a genetic defect in a lipodystrophy gene that prevents or impairs the generation of adipose tissue. Lipodystrophy genes are described above.

The metabolism of an individual suitable for treatment as described herein may display dysfunction that is characteristic of lipodystrophy. For example, the individual may display (i) elevated blood glucose levels and/or hyperglycaemia; (ii) fatty liver disease; (iii) hepatomegaly; (iv) elevated liver triglyceride levels; and/or (v) insulin resistance.

Individuals suitable for treatment may be of any age. In some embodiments, the individual is not prenatal or neonatal, for example the individual may be an adult.

Treatment pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition.

The viral expression vector or particle may be administered to an individual in an effective or therapeutically effect amount i.e. an amount that is sufficient to provide some improvement, benefit or desired therapeutic effect to the individual, for example, an amount that will provide some alleviation, mitigation, or decrease in at least one clinical symptom in the individual, for example at least one symptom of metabolic dysfunction. Those skilled in the art will appreciate that the therapeutic effects need not be complete or curative, as long as some benefit is provided to the individual.

The viral particles and expression vectors described herein may be delivered to the subject in a variety of ways, such as intravenously, or intraperitoneally. In some preferred embodiments, said particles and expression vectors described herein can be delivered to the subject via intravenous or intraperitoneal injection, preferably intravenous injection. The particular method and site of administration would be at the discretion of the physician who would also select administration techniques using his/her common general knowledge and those techniques known to a skilled practitioner.

In some embodiments, the viral expression vector or particle may be administered in a single dose. In other embodiments, the viral expression vector or particle may be administered in two or more doses at intervals, for example weekly, monthly, annually or biannually.

Following administration of the viral particles, the recipient individual may exhibit reduction in symptoms of lipodystrophy or metabolic dysfunction associated with lipodystrophy. For example, an individual being treated may exhibit one or more of (i) increased amounts of adipose tissue, for example visceral adipose tissue, (ii) increased or restored metabolic homeostasis in the individual, (iii) increased adipocyte development; (iv) reduced blood glucose levels and/or hyperglycaemia (v) improved or ameliorated fatty liver disease; (vi) reduced hepatomegaly and liver triglyceride levels; and/or (vii) reduced or abolished insulin resistance and/or (viii) increased life expectancy.

In some embodiments, the methods or treatments of the present invention may be combined with other therapies, whether symptomatic or disease modifying.

The term “treatment” includes combination treatments and therapies, in which two or more treatments or therapies are combined, for example, sequentially or simultaneously. For example it may be beneficial to combine treatment with a compound as described herein with one or more other (e.g., 1, 2, 3, 4) agents or therapies. For example, the methods or treatments of the present invention may be combined with administration of metformin or thiazolidinedione to facilitate hepatic function or adipose tissue development.

Appropriate examples of co-therapeutics will be known to those skilled in the art on the basis of the disclosure herein. Typically the co-therapeutic may be any known in the art which it is believed may give therapeutic effect in treating lipodystrophy or the symptoms thereof, subject to the diagnosis of the individual being treated.

The agents (i.e. viral particle, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Experiments

Adeno-Associated Virus Vector Delivery to Murine Tissues

We investigated whether AAV mediated gene therapy offers an effective form of treatment in a pre-clinical mouse model of congenital generalised lipodystrophy.

We first explored how efficiently genes of interest could be delivered to murine tissues, using different viral titers and modes of administration. A pilot study was conducted, where male C57BL/6J mice were injected with AAV8 vectors expressing eGFP driven by the cytomegalovirus (CMV) promoter (AAV-CMV-eGFP). Intraperitoneal (I.P.) or intravenous (I.V.) injections were made using 1×10¹⁰, 1×10¹¹ or 1×10¹² genome copies of AAV-CMV-eGFP or an equivalent volume of PBS, which was injected as a control. Mice were left for two weeks and then tissues were collected.

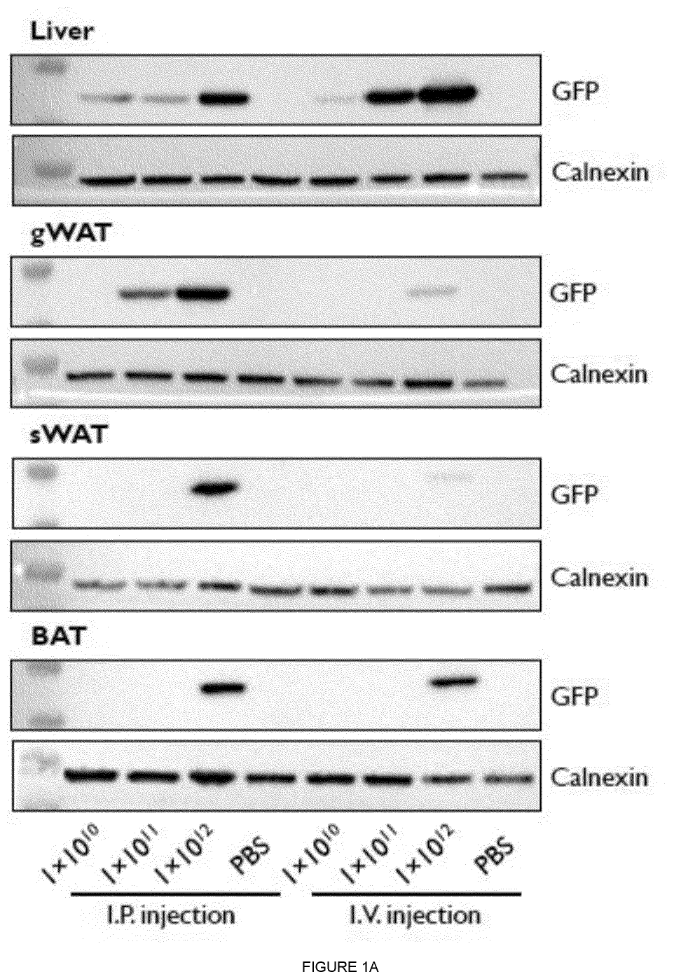

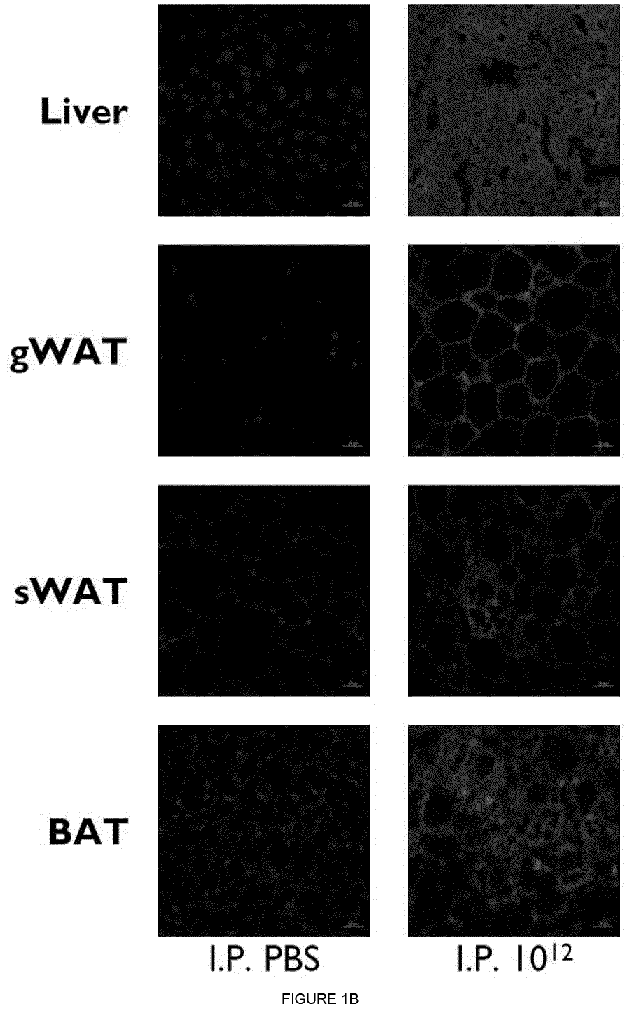

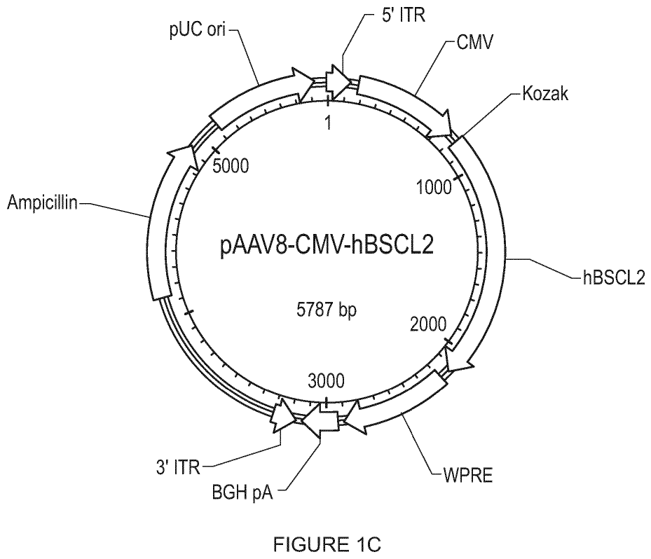

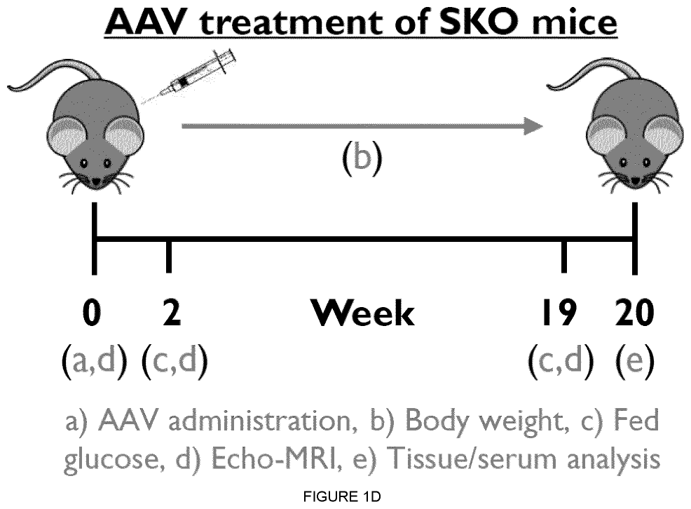

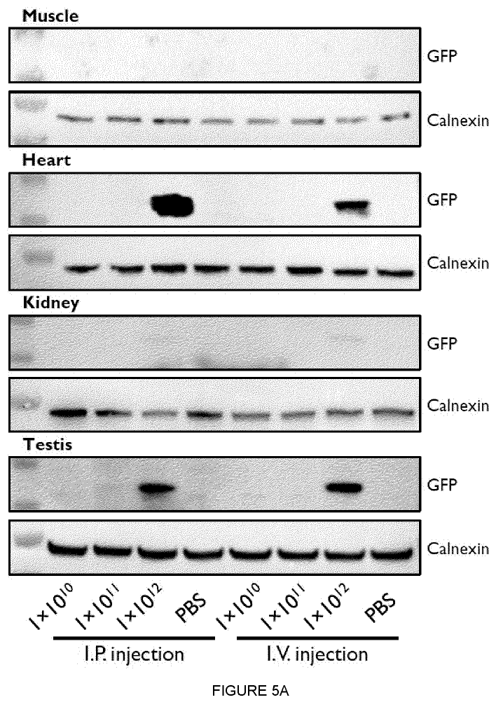

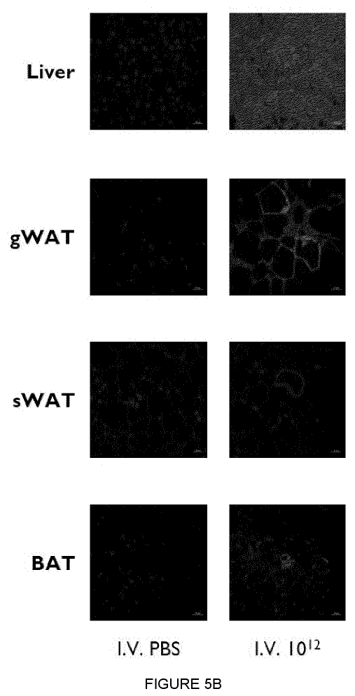

Western blot analysis revealed eGFP expression was detectable in the heart and testis, but only at a dose of 1×10¹² by I.P. or I.V. injection. Little or no expression was observed in the muscle or kidney (FIG. 5A). Within the liver however, eGFP expression was present at all doses examined, with stronger expression being observed using the I.V. route (FIG. 1A). We next examined eGFP expression in different adipose tissue depots. At a dose of 1×10¹², eGFP was detectable in gonadal white adipose tissue (gWAT), subcutaneous white adipose tissue (sWAT) and brown adipose tissue (BAT) by I.P. injection. In contrast to the liver, I.P. administration produced stronger eGFP expression within adipose tissue depots. Indeed, expression was observed in gWAT at a dose of 1×10¹¹ viral genome copies by I.P (FIG. 1A). Adipose tissue targeting by I.P. and I.V. administration was confirmed when eGFP protein levels were examined by immunohistochemistry (FIG. 1B & FIG. 5B).

Having shown that the CMV promoter is capable of effectively targeting both the liver and adipose tissues, we developed a gene therapeutic rescue strategy to treat a mouse model of congenital generalised lipodystrophy. We designed an AAV8 vector to overexpress the long isoform of the human BSCL2 gene, driven by the CMV promoter (AAV8-CMV-hBSCL2). This was commercially sourced and produced to achieve viral vectors with high titer, purity and potency (FIG. 1C). We then generated cohorts of 10-14-week-old male and female seipin knockout (SKO) mice, which our laboratory has recently established. SKO mice were randomised into two groups and injected with 1×10¹² genome copies of AAV8-CMV-hBSCL2 (SKO-hBSCL2) or AAV8-CMV-eGFP (SKO-eGFP) by I.P. administration. Mice were then maintained on a chow diet and monitored over a twenty-week period. Physiological and metabolic measurements were performed to determine if gene therapy was capable of rescuing severe metabolic disease within this pre-clinical mouse model of congenital generalised lipodystrophy (FIG. 1D).

Gene Therapy Prevents Weight Gain and Rescues Hyperglycaemia in Seipin Knockout Mice

Having injected SKO mice with AAV vectors overexpressing hBSCL2 or eGFP, we monitored body weight gain over a twenty-week period and compared this to wild type (WT) littermate controls. Weight gain in both WT and SKO-eGFP mice increased by approximately 25-30% compared to levels prior to AAV administration. No significant differences were observed at any point between WT and SKO-eGFP.

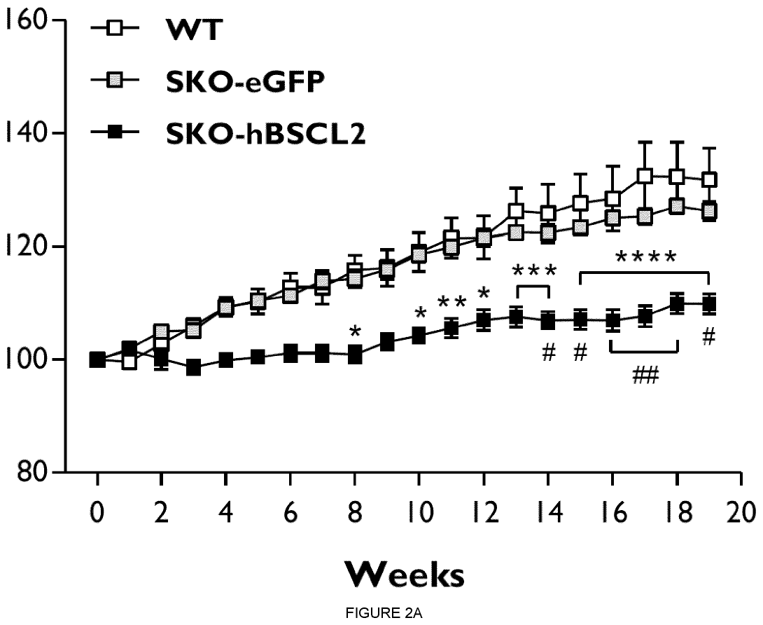

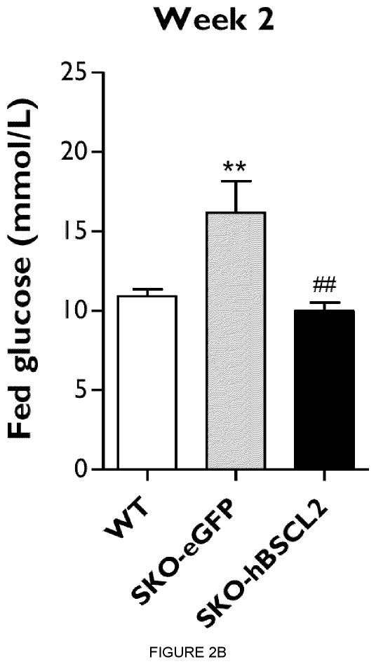

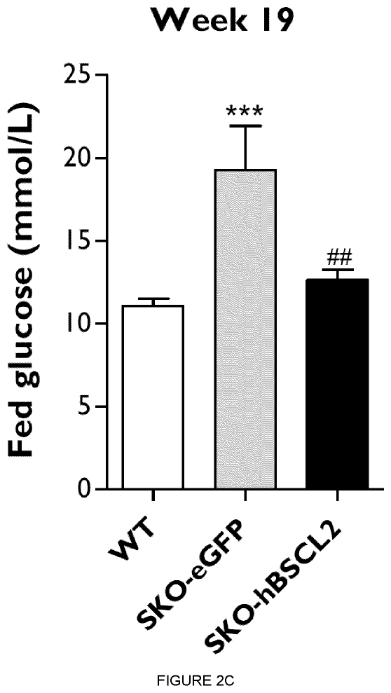

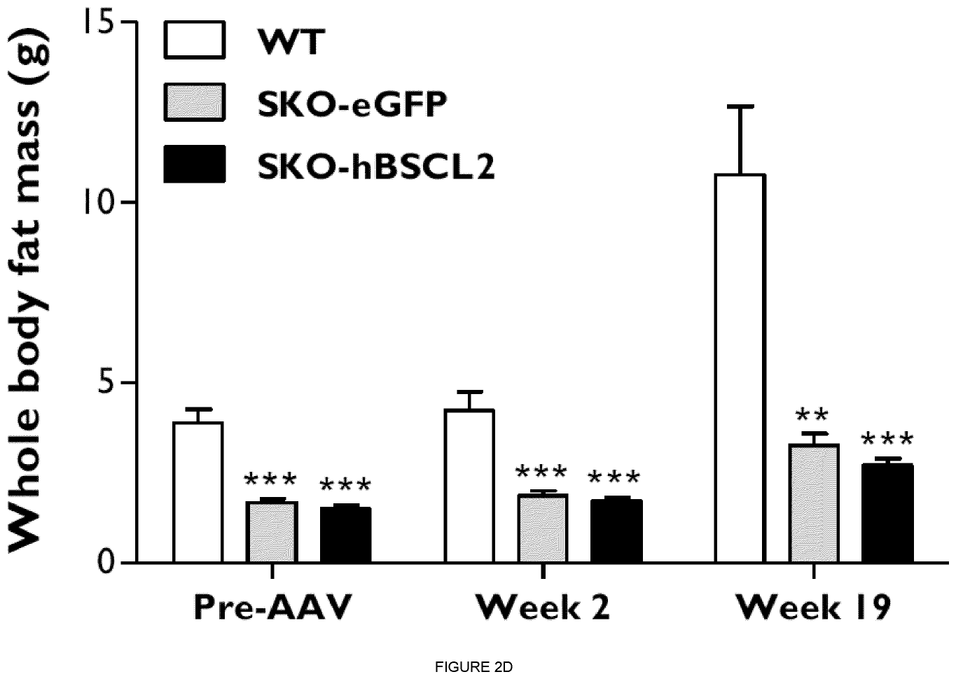

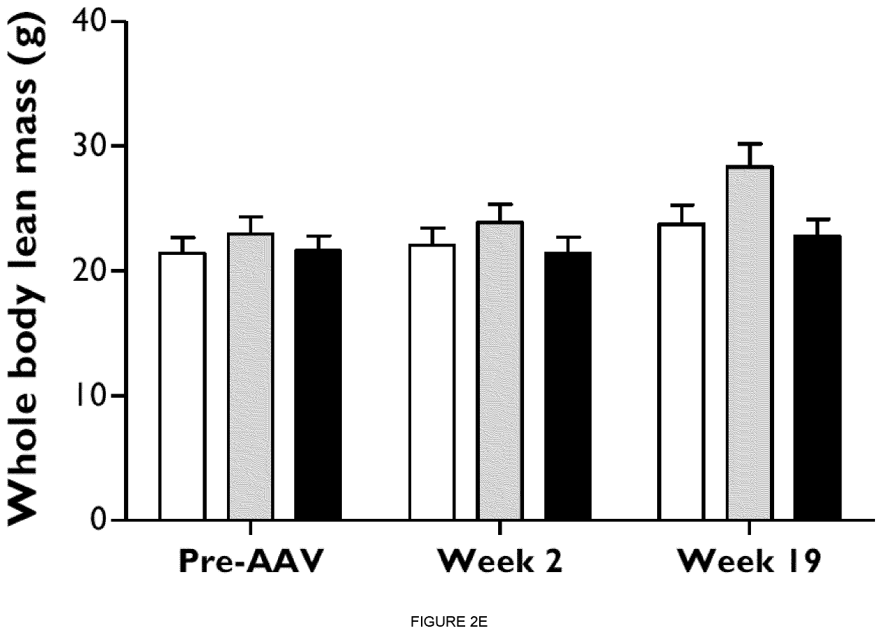

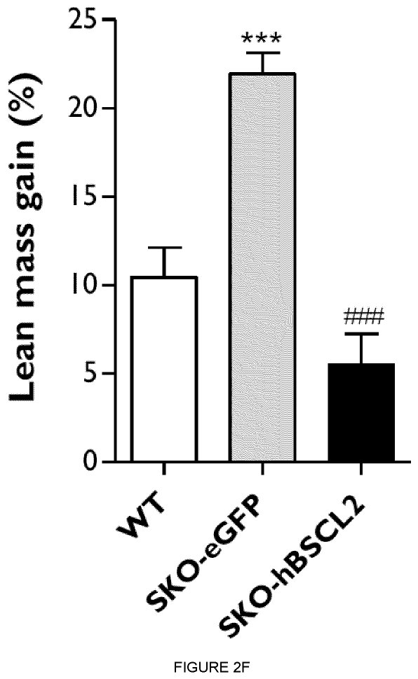

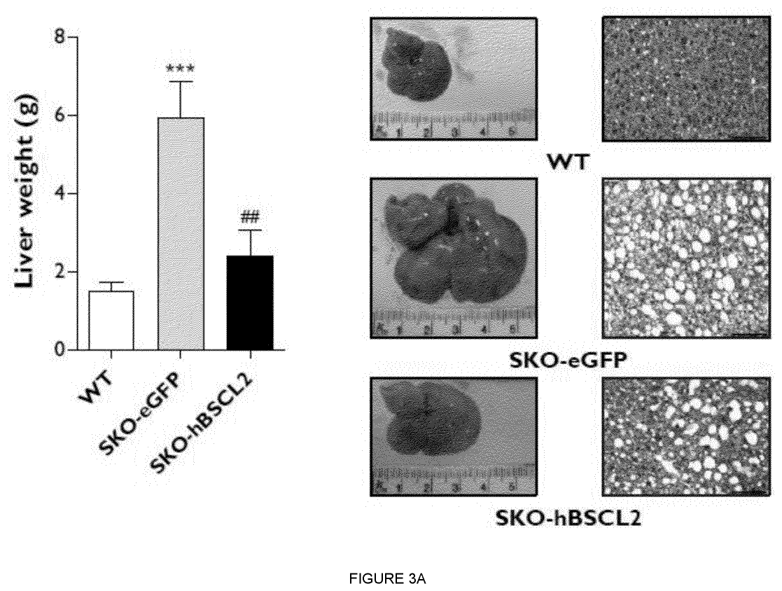

Surprisingly however, SKO-hBSCL2 mice resisted similar increases in weight gain, which were apparent as early as three weeks after treatment. This effect persisted for the duration of the experiment, with significant differences being observed in SKO-hBSCL2 mice compared to both WT and SKO-eGFP controls from week 14 onwards (FIG. 2A). Examination of blood glucose levels in ad lib fed mice indicated that SKO-eGFP mice were hyperglycaemic compared to WT controls. This was significantly rescued in SKO-hBSCL2 mice after only two weeks of treatment (FIG. 2B). Impressively, this significant improvement to glucose homeostasis was still present in SKO-hBSCL2 five months after a single intervention with gene therapy (FIG. 2C).

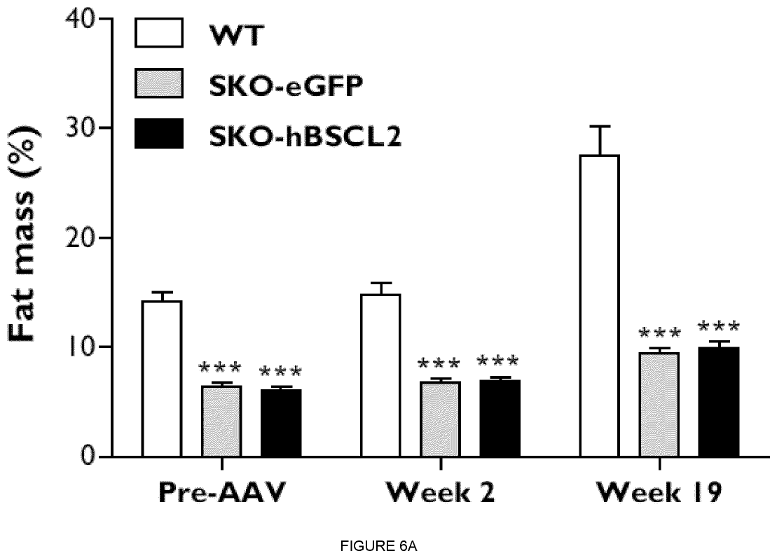

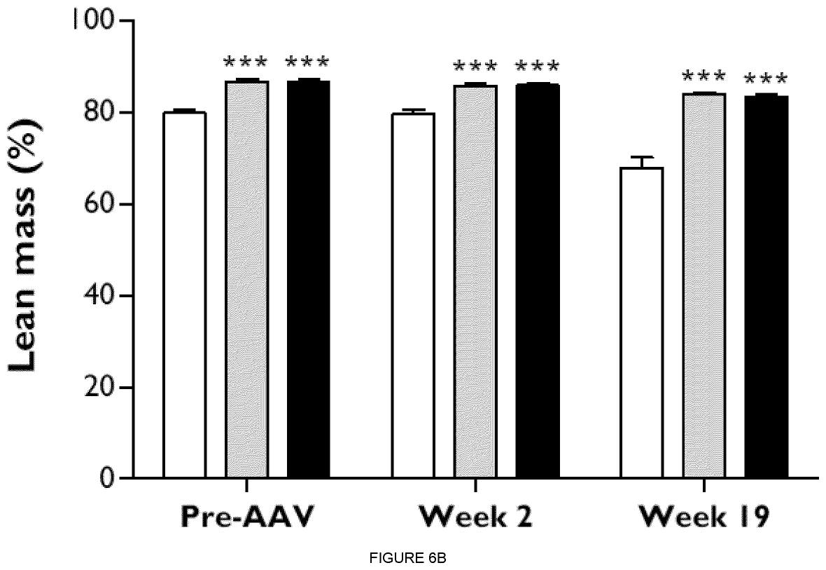

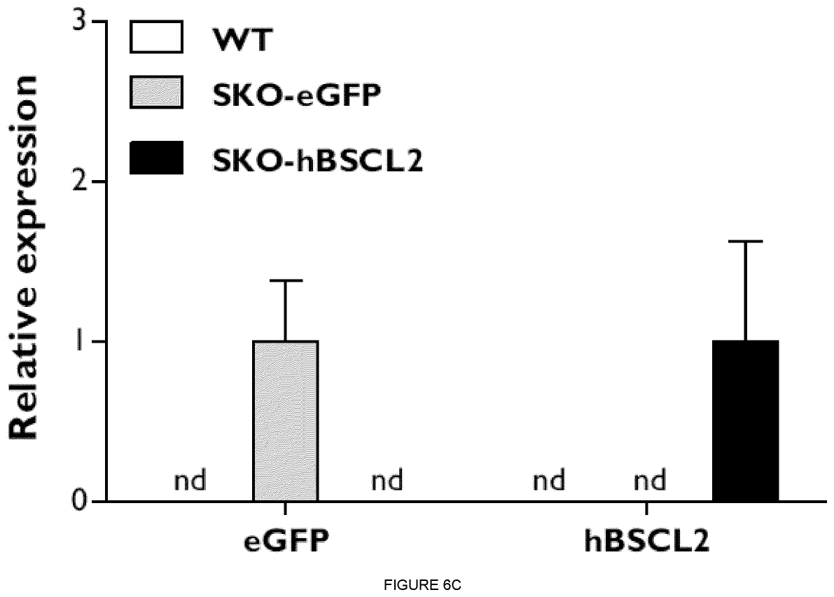

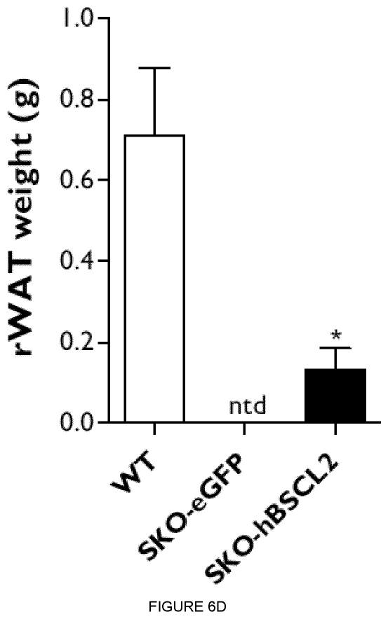

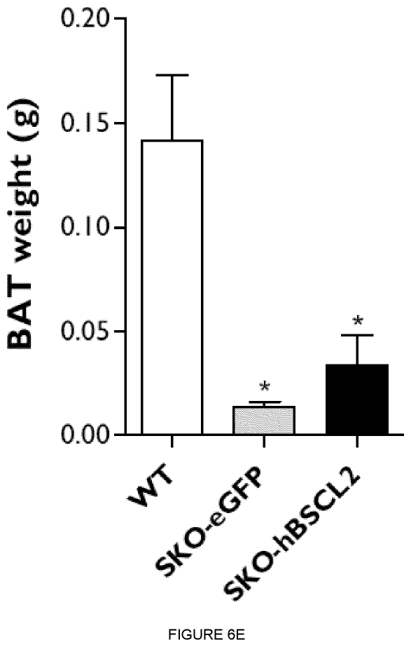

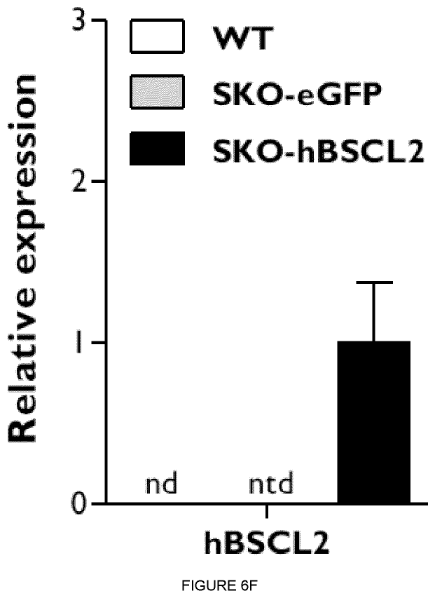

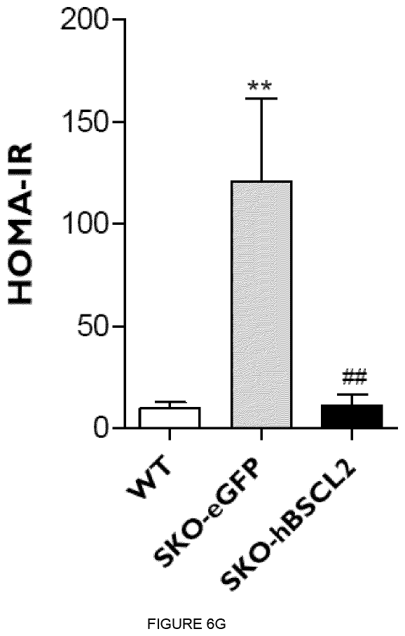

We performed Echo-MRI analysis to examine if any changes to body composition were apparent that may explain the improvements in glucose homeostasis. Prior to AAV administration, SKO mice had significantly decreased whole body fat mass compared to WT controls (FIG. 2D). At two and nineteen weeks after administration of gene therapy, whole body fat levels remained significantly decreased compared to WT controls. No significant differences in whole body fat content were detectable between SKO-eGFP and SKO-hBSCL2 at any time point (FIG. 2D). Similar results were observed when whole body fat levels were normalised to body weight and represented as a percentage (FIG. 6A). Due to the significantly decreased body fat mass in SKO mice, whole body lean mass levels normalised to body weight were significantly increased in SKO-eGFP and SKO-hBSCL2 mice compared to WT controls. This was present at all time points and no significant differences were found when comparing SKO-eGFP and SKO-hBSCL2 mice (FIG. 6B). When we examined absolute whole-body lean mass levels, this appeared to have increased in SKO-eGFP but not SKO-hBSCL2 mice at week nineteen, but this was not significantly different (FIG. 2E). As both male and female mice were being examined, we calculated the percentage of lean mass gained during the experiment for each animal. We found that SKO-eGFP mice gained significantly more lean mass compared to WT mice over the nineteen-week period. SKO-hBSCL2 mice however failed to similarly increase their lean mass levels, which were significantly lower compared to SKO-eGFP mice (FIG. 2F).

Our findings therefore indicate that a single administration of AAV mediated gene therapy is capable of producing rapid and prolonged improvements to glucose homeostasis in SKO mice. These alterations appear to be associated with the significant changes to lean mass levels observed in SKO mice, rather than alterations to whole body fat content.

Gene Therapy Prevents Hepatomegaly and Restores Visceral Adipose Tissue Development

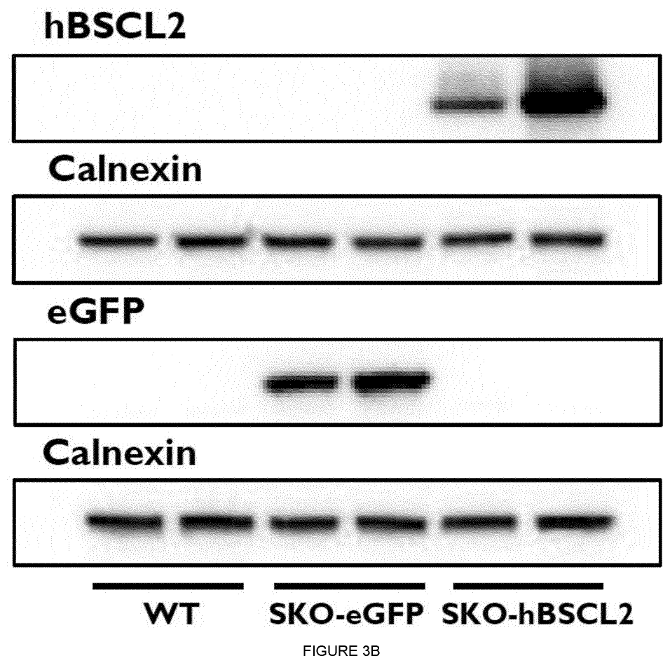

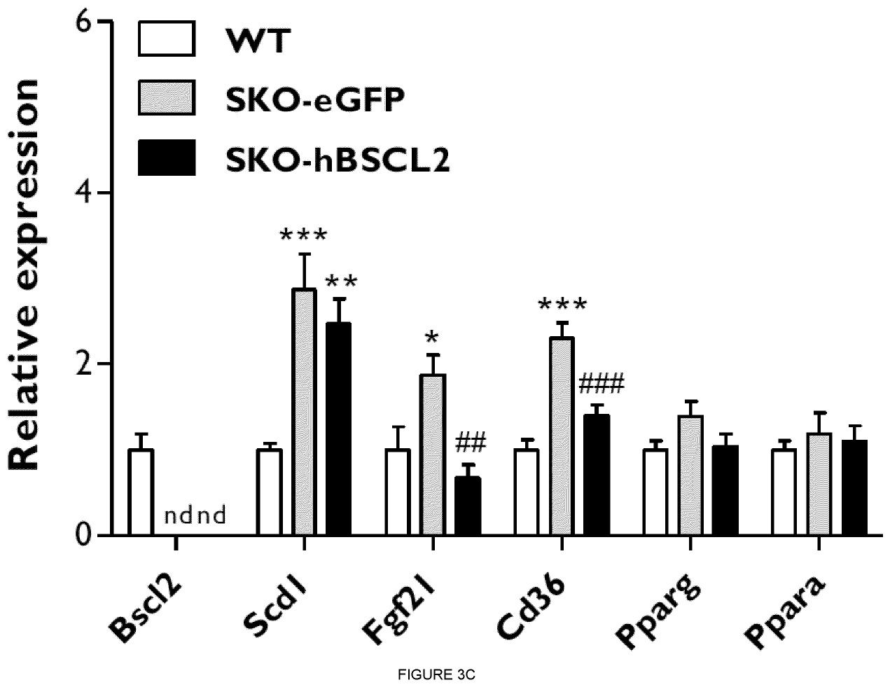

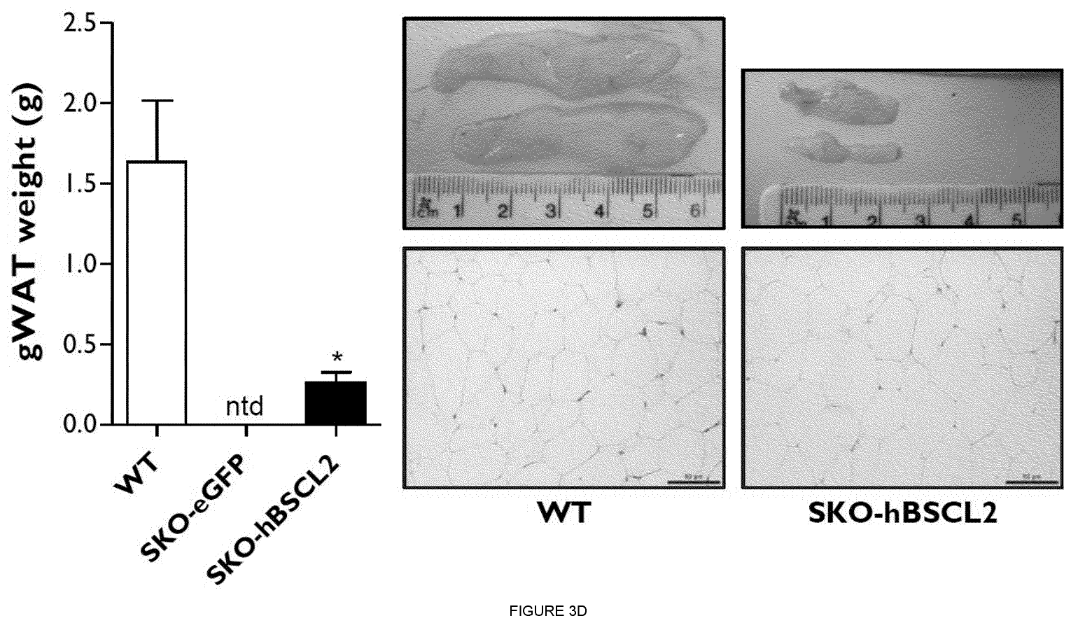

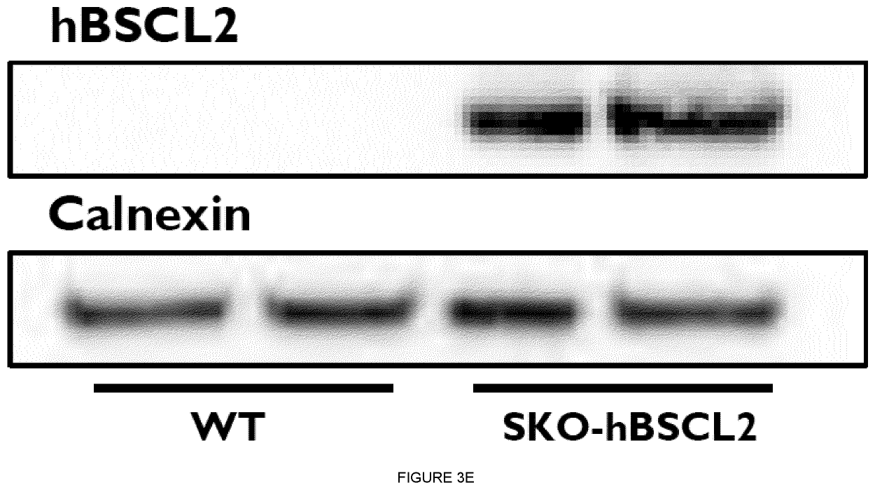

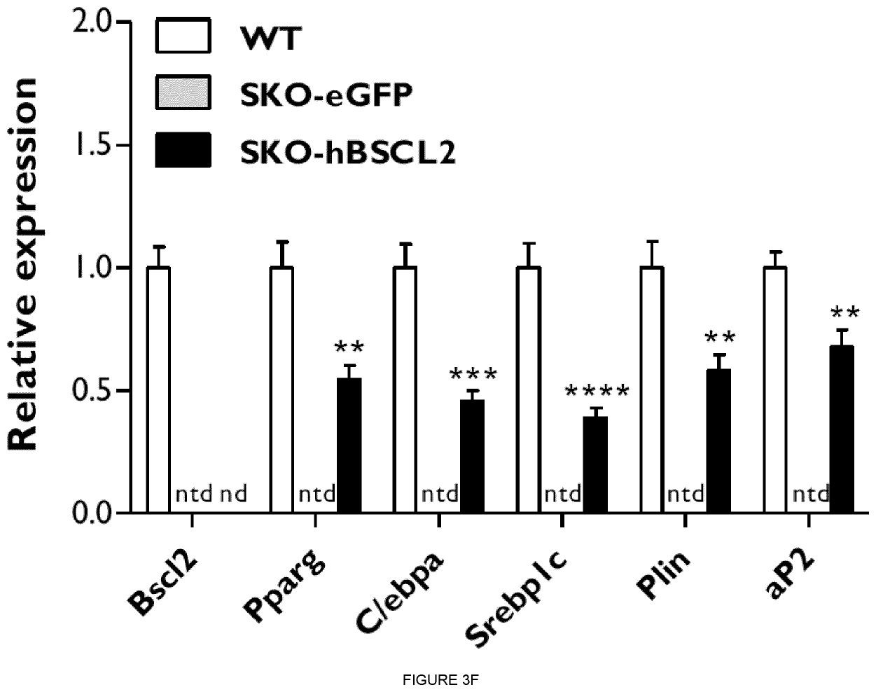

A key metabolic feature observed in SKO mice is hepatomegaly. This is caused by the lack of adipose tissue development, leading to ectopic accumulation of lipids in the liver. As expected, SKO-eGFP mice had significantly increased liver weights compared to WT controls. Despite only receiving a single treatment, liver weights in SKO-hBSCL2 mice were significantly decreased compared to SKO-eGFP controls five months after treatment. H&E staining also indicated lipid accumulation appeared reduced in SKO-hBSCL2 mice, however was still present (FIG. 3A). Western blot analysis indicated that both eGFP and hBSCL2 protein levels were detectable in the livers of these mice at the termination of the experiment (FIG. 3B). This was also confirmed by examining mRNA transcript levels of eGFP and hBSCL2 in the liver (FIG. 6C). We next examined hepatic markers that have previously been shown to be dysregulated in SKO mice (1, 2). We confirmed that expression of the mouse BscI2 gene was absent in SKO mice. The de novo lipogenesis marker Scd1 was significantly upregulated in SKO mice compared to WT controls. Treatment with gene therapy had no effect on the expression level of this marker (FIG. 3C). We found no significant differences when we examined Pparg or Ppara expression levels in each of the conditions examined. Expression levels of Fgf21 and the fatty acid transporter Cd36 were found to be significantly increased in SKO-eGFP mice compared to WT controls. Interestingly, both markers were normalised in SKO-hBSCL2 mice, with expression levels significantly decreased compared to SKO-eGFP mice (FIG. 3C).

Having observed no significant alterations in whole body fat content between SKO-eGFP and SKO-hBSCL2 mice (FIG. 2D), we were surprised to discover that gene therapy led to the substantial development of visceral adipose tissues in SKO-hBSCL2 mice. We were able to dissect adipose tissue from retroperitoneal (FIG. 6D) and gonadal (FIG. 3D) depots from SKO-hBSCL2 mice, which were completely absent in SKO-eGFP mice. H&E stained adipose tissue sections from gWAT of SKO-hBSCL2 mice were similar to those observed in WT mice (FIG. 3D). Small amounts of BAT were detectable in SKO mice, which were significantly reduced compared to WT mice and not significantly changed by AAV intervention (FIG. 6E). Similar to that observed in the liver, western blot analysis revealed hBSCL2 protein was detectable in the rescued gWAT five months after intervention (FIG. 3E). This was also verified by qPCR analysis of hBSCL2 transcript levels (FIG. 6F).

We also examined expression levels of key transcription factors (Pparg, C/ebpa and Srebp1c) and markers of adipogenesis (Plin and aP2) within the rescued gWAT of SKO-hBSCL2 mice. We again confirmed the complete lack of mouse BscI2 gene expression present in restored gWAT of SKO-hBSCL2 mice. Substantial expression was evident for all adipogenic markers examined in SKO-hBSCL2 mice, however the levels were significantly lower than those detected in WT mice (FIG. 3F).

Overall, our findings indicate that a single intervention of gene therapy is able to substantially restore the development of visceral WAT depots in adult SKO mice. These adipose tissue depots were maintained for a period of five months and is likely to be responsible for the improvements in hepatomegaly and glucose homeostasis detected within this model.

Insulin Sensitivity is Restored in Seipin Knockout Mice Administered with Gene Therapy

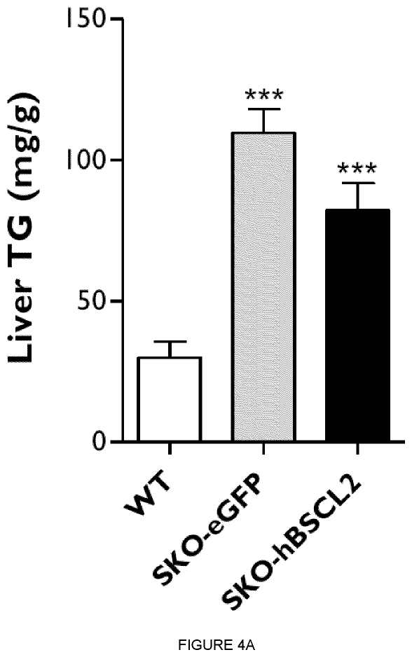

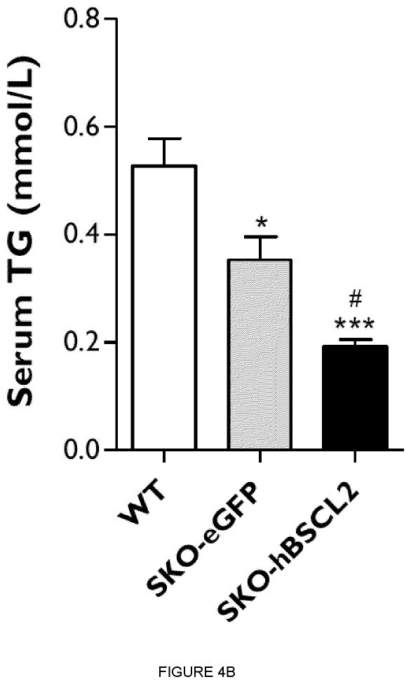

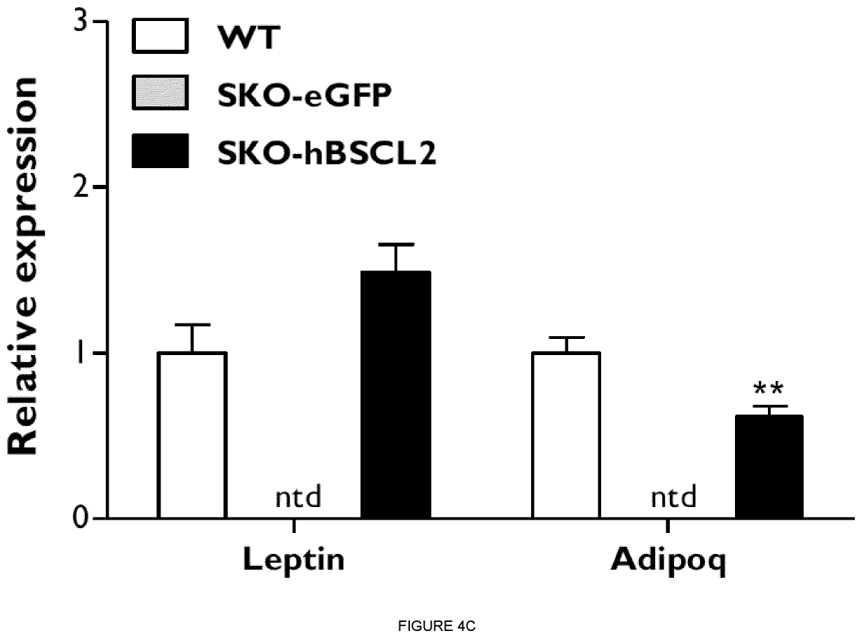

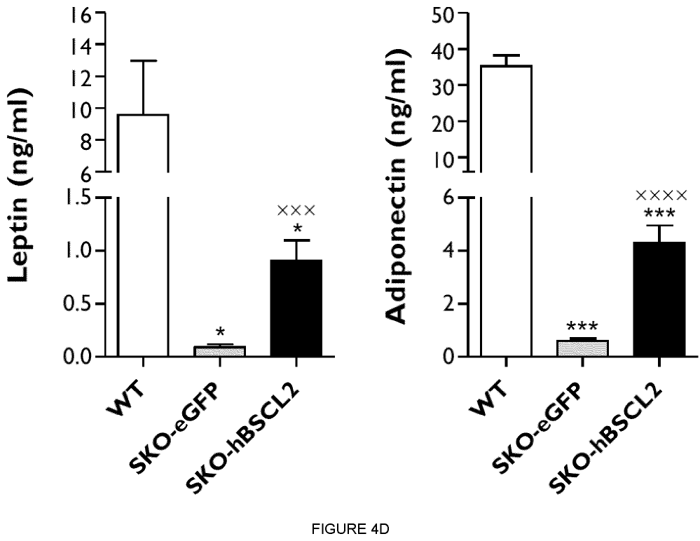

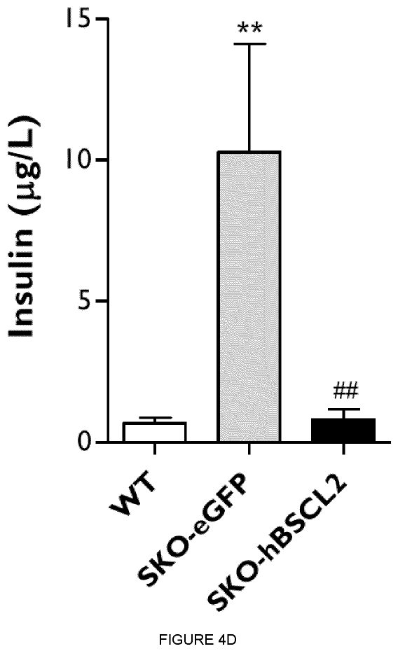

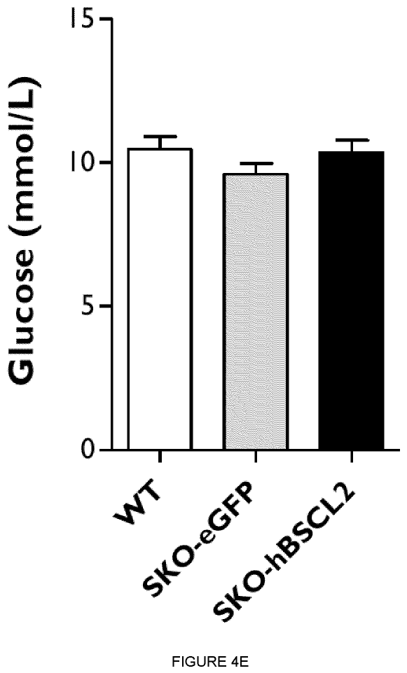

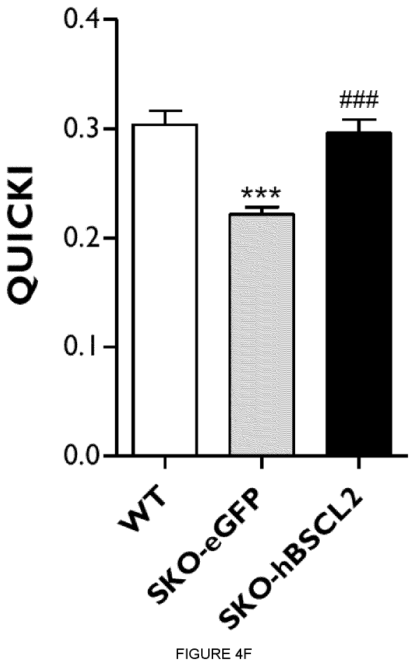

To determine if gene therapy had any additional beneficial effects, we examined if other metabolic complications that develop in conditions of lipodystrophy had been improved. Despite the reductions we observed in liver weights of SKO-hBSCL2 mice (FIG. 2A), hepatic triglyceride (TG) content per mg of tissue in SKO-eGFP and SKO-hBSCL2 remained significantly elevated compared to WT control mice. Whilst lipid levels did appear to be lower in SKO-hBSCL2 mice compared to SKO-eGFP controls, this was not found to be significantly different (FIG. 4A). Serum TG levels were found to be significantly decreased in SKO-eGFP mice fasted for five hours, a feature that has been observed previously (3-5). However, gene therapy significantly decreased serum TG levels further in SKO-hBSCL2 mice (FIG. 4B).

We next examined whether the rescue of visceral WAT resulted in any alterations to important adipokines such as leptin and adiponectin. Examination of gene expression in gWAT revealed that leptin expression was restored to levels not significantly different to WT control mice. Adiponectin expression was also readily detectable in SKO-hBSCL2 mice, although this was significantly lower than WT controls (FIG. 4C). When circulating serum leptin and adiponectin levels were analysed, both leptin and adiponectin were significantly decreased in SKO-eGFP mice compared to WT controls. Only modest increases in circulating leptin and adiponectin levels were apparent in SKO-hBSCL2 mice, which remained significantly lower compared to WT mice. However, when SKO-eGFP and SKO-hBSCL2 mice were compared directly, both adipokines were found to be significantly increased in response to administration of gene therapy (FIG. 4D).

We finally examined whether gene therapy could prevent insulin resistance in SKO mice. Examination of fasted circulating insulin levels revealed significant elevations in SKO-eGFP mice compared to WT controls. Impressively, gene therapy restored fasting insulin levels in SKO-hBSCL2 mice to levels not significantly different to WT controls (FIG. 4E). Fasting circulating glucose levels were not found to be significantly different between any of the conditions (FIG. 4F). When we performed quantitative insulin-sensitivity check index (QUICKI) analysis, as anticipated, SKO-eGFP mice had significantly decreased values compared to WT mice, indicating insulin resistance. Gene therapy completely normalised this in SKO-hBSCL2 mice (FIG. 4G). Similar findings were also apparent when homeostatic model assessment of insulin resistance (HOMA-IR) was performed (FIG. 6G).

Overall, our findings reveal that gene therapy can restore multiple metabolic complications that arise in conditions of lipodystrophy, which manifest due to the failure of appropriate adipose tissue development. Importantly, a single intervention was capable of restoring glucose homeostasis and insulin sensitivity within our pre-clinical mouse model of CGL2.

| Reference Sequences | |
|---|---|
| 1 | atttgaaaat ctgacatcag ctgggcagtc gcccccctcc tcctttcctc cctctactct |
| 61 | gacacagcac ttagcacctg aatcttcgtt tctctcccag ggaccctcca ttttccatat |
| 121 | ccaggaaaat gtgatgcgcc acaggtatca gcgtctggat cgccacttca cgttttagcc |
| 181 | acaagtgact cagtggaaga tccagagtca acagaggctc gtcaggaaga tgtctacaga |
| 241 | aaaggtagac caaaaggagg aagctgggga aaaagaggtg tgcggagacc agatcaaagg |
| 301 | accggacaaa gaggaggaac caccagctgc tgcatcccat ggccaggggt ggcgtccagg |
| 361 | tggcagagca gctaggaacg caaggcctga acctggggcc agacaccctg ctctcccggc |
| 421 | catggtcaac gaccctccag tacctgcctt actgtgggcc caggaggtgg gccaagtctt |
| 481 | ggcaggccgt gcccgcaggc tgctgctgca gtttggggtg ctcttctgca ccatcctcct |
| 541 | tttgctctgg gtgtctgtct tcctctatgg ctccttctac tattcctata tgccgacagt |
| 601 | cagccacctC agccctgtgc atttctacta caggaccgac tgtgattcct ccaccacctc |
| 661 | actctgctcc ttccctgttg ccaatgtctc gctgactaag ggtggacgtg atcgggtgct |
| 721 | gatgtatgga cagccgtatc gtgttacctt agagcttgag ctgccagagt cccctgtgaa |
| 781 | tcaagatttg ggcatgttct tggtcaccat ttcctgctac accagaggtg gccgaatcat |
| 841 | ctccacttct tcgcgttcgg tgatgctgca ttaccgctca gacctgctcc agatgctgga |
| 901 | cacactggtc ttctctagcc tcctgctatt tggctttgca gagcagaagc agctgctgga |
| 961 | ggtggaactc tacgcagact atagagagaa ctcgtacgtg ccgaccactg gagcgatcat |
| 1021 | tgagatccac agcaagcgca tccagctgta tggagcctac ctccgcatcc acgcgcactt |
| 1081 | cactgggctc agatacctgc tatacaactt cccgatgacc tgcgccttca taggtgttgc |
| 1141 | cagcaacttc accttcctca gcgtcatcgt gctcttcagc tacatgcagt gggtgtgggg |
| 1201 | gggcatctgg ccccgacacc gcttctcttt gcaggttaac atccgaaaaa gagacaattc |
| 1261 | ccggaaggaa gtccaacgaa ggatctctgc tcatcagcca gggcctgaag gccaggagga |
| 1321 | gtcaactccg caatcagatg ttacagagga tggtgagagc cctgaagatc cctcagggac |
| 1381 | agagggtcag ctgtccgagg aggagaaacc agatcagcag cccctgagcg gagaagagga |
| 1441 | gctagagcct gaggccagtg atggttcagg ctcctgggaa gatgcagctt tgctgacgga |
| 1501 | ggccaacctg cctgctcctg ctcctgcttc tgcttctgcc cctgtcctag agactctggg |
| 1561 | cagctctgaa cctgctgggg gtgctctccg acagcgcccc acctgctcta gttcctgaag |
| 1621 | aaaaggggca gactcctcac attccagcac tttcccacct gactcctctc ccctcgtttt |
| 1681 | tccttcaata aactattttg tgtcagcttc |
| BSCL2 coding sequence | |
| (SEQ ID NO: 1; NM_001122955.4) | |
| 001 | mstekvdqke eagekevcgd qikgpdkeee ppaaashgqg wrpggraarn arpepgarhp |
| 061 | alpamvndpp vpallwaqev gqvlagrarr lllqfgvlfc tillllwvsv flygsfyysy |
| 121 | mptvshlspv hfyyrtdcds sttslcsfpv anvsltkggr drvlmygqpy rvtlelelpe |
| 181 | spvnqdlgmf lvtiscytrg griistssrs vmlhyrsdll qmldtlvfss lllfgfaeqk |
| 241 | qllevelyad yrensyvptt gaiieihskr iqlygaylri hahftglryl lynfpmtcaf |
| 301 | igvasnftfl svivlfsymq wvwggiwprh rfslqvnirk rdnsrkevqr risahqpgpe |
| 361 | gqeestpqsd vtedgesped psgtegqlse eekpdqqpls geeelepeas dgsgswedaa |
| 421 | llteanlpap apasasapvl etlgssepag galrqrptcs ss |
| BSCL2 amino acid sequence | |
| (SEQ ID NO: 2; NP_001116427.1) | |
| 1 | agccccgccg ccctcgcaat aaggggcctg agcgcgcggg ggagaagcgg gagcgggagc |
| 61 | gggagcgagc tggcggcgcc gtcgggcgcc gggccgggcc atggagctgt ggccgtgtct |
| 121 | ggccgcggcg ctgctgttgc tgctgctgct ggtgcagctg agccgcgcgg ccgagttcta |
| 181 | cgccaaggtc gccctgtact gcgcgctgtg cttcacggtg tccgccgtgg cctcgctcgt |
| 241 | ctgcctgctg cgccacggcg gccggacggt ggagaacatg agcatcatcg gctggttcgt |
| 301 | gcgaagcttc aagtactttt acgggctccg cttcgaggtg cgggacccgc gcaggctgca |
| 361 | ggaggcccgt ccctgtgtca tcgtctccaa ccaccagagc atcctggaca tgatgggcct |
| 421 | catggaggtc cttccggagc gctgcgtgca gatcgccaag cgggagctgc tcttcctggg |
| 481 | gcccgtgggc ctcatcatgt acctcggggg cgtcttcttc atcaaccggc agcgctctag |
| 541 | cactgccatg acagtgatgg ccgacctggg cgagcgcatg gtcagggaga acgtgcccat |
| 601 | cgtccccgtg gtgtactctt ccttctcctc cttctacaac accaagaaga agttcttcac |
| 661 | ttcaggaaca gtcacagtgc aggtgctgga agccatcccc accagcggcc tcactgcggc |
| 721 | ggacgtccct gcgctcgtgg acacctgcca ccgggccatg aggaccacct tcctccacat |
| 781 | ctccaagacc ccccaggaga acggggccac tgcggggtct ggcgtgcagc cggcccagta |
| 841 | gcccagacca cggcagggca tgacctgggg agggcaggtg gaagccgatg gctggaggat |
| 901 | gggcagaggg gactcctccc ggcttccaaa taccactctg tccggctccc ccagctctca |
| 961 | ctcagcccgg gaagcaggaa gccccttctg tcactggcct cagacacagg cccctggtgt |
| 1021 | cccctgcagg gggctcagct ggaccctccc cgggctcgag ggcagggact cgcgcccacg |
| 1081 | gcacctctgg gagctgggat gataaagatg aggcttgcgg ctgtggcccg ctggtgggct |
| 1141 | gagccacaag gcccccgatg gcccaggagc agatgggagg accccgaggc cagacgcaca |
| 1201 | ctgtccgagc cctctgctca gccgcctggg acccaccagg gtgcagctgg gctccagggt |
| 1261 | ccagcccaca agctgcatca ggctctctgg gagaggaggg gcctggaggg ccaggagtcc |
| 1321 | cagactcacg caccctgggc cacagggagc cgggaatcgg ggcctgctgc tcctgctggc |
| 1381 | ctggaagact ctgtggggtc agcactgtac tccgttgctg tttttttata aacacactct |
| 1441 | tggaagtggc |
| AGPAT2 coding sequence | |
| (SEQ ID NO: 3; NM_001012727.2) | |
| 001 | melwpclaaa lllllllvql sraaefyakv alycalcftv savaslvcll rhggrtvenm |
| 061 | siigwfvrsf kyfyglrfev rdprrlqear pcvivsnhqs ildmmglmev lpercvqiak |
| 121 | rellflgpvg limylggvff inrqrsstam tvmadlgerm vrenvpivpv vyssfssfyn |
| 181 | tkkkfftsgt vtvqvleaip tsgltaadvp alvdtchram rttflhiskt pqengatags |
| 241 | gvqpaq |
| AGPAT2 amino acid sequence | |
| (SEQ ID NO: 4; NP_001012745.1) | |
| 001 | taaagcacag cccagggaaa cctcctcaca gttttcatcc agccacgggc cagcatgtct |
| 061 | gggggcaaat acgtagactc ggaggtaggc atccgtgggg gggcgccggc tcgggcgtgc |
| 121 | ggggagtgtc cgcttctgct atctgcctct ccaaatatcc cgactgctgc cctggcccca |
| 181 | gccctctctc cacttcggag cactcctctg gcgttggcac cgctgaggaa tgggcctggg |
| 241 | cggggaggtg aagagaagcc aggaatgttt tatgttttcc taatggagag ggggcctagg |
| 301 | gagcccctga gctaggagga cacggaaaag gggattgggg tcctgagatt gggtctgttg |
| 361 | ggcccaggac gcgttttctg gatgggtcta ggatgctccc ttgtcgcggg acccccgcgg |
| 421 | tccggccctg cctgctgggg gttcgaagag gtggagtgca gggtggaggt gttatttacc |
| 481 | cgagtcctgg ggacagtccc cgggactctc cgccaggcgc ccagaccggc aggtcccgca |
| 541 | ggcggcgcgc ggtgtgtttg cactttccaa agttcttgaa ccatctcaag aactccttct |
| 601 | gcatcttggc gtctggcagg ggtgttccga gagaggtaga cctcccctcc ccaaactgcc |
| 661 | accatcactt ccaacgccct ccacgcgctg gagctctgcc cgggtgtgga aacctcgtct |
| 721 | tccaacacgt agctgccctt cagccacccg cccgcagcct gggagtgccc tgagggtggg |
| 781 | tcgggggagc tgcgcaggga catctctaca ccgttcccat ccgggaacag ggcaacatct |
| 841 | acaagcccaa caacaaggcc atggcagacg agctgagcga gaagcaagt tacgacgcgc |
| 901 | acaccaagga gatcgacctg gtcaaccgcg accctaaaca cctcaacgat gacgtggtca |
| 961 | agattgactt tgaagatgtg attgcagaac cagaagggac acacagtttt gacggcattt |
| 1021 | ggaaggccag cttcaccacc ttcactgtga cgaaatactg gttttaccgc ttgctgtctg |
| 1081 | ccctctttgg catcccgatg gcactcatct ggggcattta cttcgccatt ctctctttcc |
| 1141 | tgcacatctg ggcagttgta ccatgcatta agagcttcct gattgagatt cagtgcatca |
| 1201 | gccgtgtcta ttccatctac gtccacaccg tctgtgaccc actctttgaa gctgttggga |
| 1261 | aaatattcag caatgtccgc atcaacttgc agaaagaaat ataaatgaca tttcaaggat |
| 1321 | agaagtatac ctgatttttt ttccttttaa ttttcctggt gccaatttca agttccaagt |
| 1381 | tgctaataca gcaacaattt atgaattgaa ttatcttggt tgaaaataaa aagatcactt |
| 1441 | tctcagtttt cataagtatt atgtctcttc tgagctattt catctatttt tggcagtctg |
| 1501 | aatttttaaa acccatttaa atttttttcc ttaccttttt atttgcatgt ggatcaacca |
| 1561 | tcgctttatt ggctgagata tgaacatatt gttgaaaggt aatttgagag aaatatgaag |
| 1621 | aactgaggag gaaaaaaaaa aaaaagaaaa gaaccaacaa cctcaactgc ctactccaaa |
| 1681 | atgttggtca ttttatgtta agggaagaat tccagggtat ggccatggag tgtacaagta |
| 1741 | tgtgggcaga ttttcagcaa actcttttcc cactgtttaa ggagttagtg gattactgcc |
| 1801 | attcacttca taatccagta ggatccagtg atccttacaa gttagaaaac ataatcttct |
| 1861 | gccttctcat gatccaacta atgccttact cttcttgaaa ttttaaccta tgatattttc |
| 1921 | tgtgcctgaa tatttgttat gtagataaca agacctcagt gccttcctgt ttttcacatt |
| 1981 | ttccttttca aatagggtct aactcagcaa ctcgctttag gtcagcagcc tccctgaaga |
| 2041 | ccaaaattag aatatccatg acctagtttt ccatgcgtgt ttctgactct gagctacaga |
| 2101 | gtctggtgaa gctcacttct gggcttcatc tggcaacatc tttatccgta gtgggtatgg |
| 2161 | ttgacactag cccaatgaaa tgaattaaag tggaccaata gggctgagct ctctgtgggc |
| 2221 | tggcagtcct ggaagccagc tttccctgcc tctcatcaac tgaatgaggt cagcatgtct |
| 2281 | attcagcttc gtttattttc aagaataatc acgctttcct gaatccaaac taatccatca |
| 2341 | ccggggtggt ttagtggctc aacattgtgt tcccatttca gctgatcagt gggcctccaa |
| 2401 | ggaggggctg taaaatggag gccattgtgt gagcctatca gagttgctgc aaacctgacc |
| 2461 | cctgctcagt aaagcacttg caaccgtctg ttatgctgtg acacatggcc cctccccctg |
| 2521 | ccaggagctt tggacctaat ccaagcatcc ctttgcccag aaagaagatg ggggaggagg |
| 2581 | cagtaataaa aagattgaag tattttgctg gaataagttc aaattcttct gaactcaaac |
| 2641 | tgaggaattt cacctgtaaa cctgagtcgt acagaaagct gcctggtata tccaaaagct |
| 2701 | ttttattcct cctgctcata ttgtgattct gcctttgggg acttttctta aaccttcagt |
| 2761 | tatgattttt ttttcataca cttattggaa ctctgcttga tttttgcctc ttccagtctt |
| 2821 | cctgacactt taattaccaa cctgttacct actttgactt tttgcattta aaacagacac |
| 2881 | tggcatggat atagttttac ttttaaactg tgtacataac tgaaaatgtg ctatactgca |
| 2941 | tactttttaa atgtaaagat atttttatct ttatatgaag aaaatcactt aggaaatggc |
| 3001 | tttgtgattc aatctgtaaa ctgtgtattc caagacatgt ctgttctaca tagatgctta |
| 3061 | gtccctcatg caaatcaatt actggtccaa aagattgctg aaattttata tgcttactga |
| 3121 | tatattttac aattttttat catgcatgtc ctgtaaaggt tacaagcctg cacaataaaa |
| 3181 | atgtttaacg gttaaacagt caaaaaaaaa aa |
| CAV1 coding sequence | |
| (SEQ ID NO: 5; NM_001172895.1) | |
| 001 | madelsekqv ydahtkeidl vnrdpkhlnd dvvkidfedv iaepegthsf dgiwkasftt |
| 061 | ftvtkywfyr llsalfgipm aliwgiyfai lsflhiwavv pciksfliei qcisrvysiy |
| 121 | vhtvcdplfe avgkifsnvr inlqkei |
| CAV1 amino acid sequence | |
| (SEQ ID NO: 6; NP_001166366.1) | |
| 0001 | agttctggcc gctgtcccgg tgcgcacgga cgtggctcga gtttcctctg ctctccgctc |
| 0061 | tcgcccgcta gctctcctcc cttccgctcc tgcttctctc cgggtctccc gctccagctc |
| 121 | cagccccacc cggccggtcc cgcacggctc cgggtagcca tggaggaccc cacgctctat |
| 181 | attgtcgagc ggccgcttcc cgggtacccc gacgccgagg ccccggagcc ttcctccgct |
| 241 | ggggctcagg cagcggagga gccgtcgggg gccggctcag aagagctgat caagtcggac |
| 301 | caggtgaacg gcgtgctggt gctgagcctc ctggacaaaa tcatcggggc cgtagaccag |
| 361 | atccagctga ctcaagcaca gctggaggag cggcaggcgg agatggaggg cgcagtgcag |
| 421 | agcatccagg gcgagctgag caagctgggc aaggcgcacg ccaccacgag caatacggtg |
| 481 | agcaagctgc tggagaaggt gcgcaaggtc agcgtcaacg tgaagaccgt gcgcggcagc |
| 541 | ctggagcgcc aggcggggca gatcaagaag ctggaggtca acgaggccga gctgctgcgg |
| 601 | cgccgcaact ttaaagtcat gatctaccag gatgaagtga agctgccggc caaactgagc |
| 661 | atcagcaaat cgctgaaaga gtcggaggcg ctgccagaga aggagggcga ggagctgggc |
| 721 | gagggcgagc ggcccgagga ggacgcagcg gcgctggagc tttcgtcgga cgaggcggtg |
| 781 | gaggttgagg aggttattga ggagtcccgc gcagagcgta tcaagcgcag cggcctgcgg |
| 841 | cgcgtggacg acttcaagaa ggccttctcc aaggagaaga tggagaagac caaggtgcgt |
| 901 | acccgcgaga acctggagaa gacgcgcctc aagaccaagg aaaacctgga gaagacgcgg |
| 961 | cacaccctgg agaagcgcat gaacaagctg ggcacgcgcc tggtgcccgc cgagcggcgc |
| 1021 | gagaaactga agacgtcgcg ggacaagttg cgcaaatcct tcacgcccga ccacgtggtg |
| 1081 | tacgcgcgct ccaagaccgc ggtctacaag gtgccaccct tcaccttcca cgtcaagaag |
| 1141 | atccgcgagg gccaggtgga agtgctcaag gccaccgaga tggtggaggt gggcgccgac |
| 1201 | gacgacgagg gcggcgcgga gcgcggggag gccggcgacc tgcggcgcgg gagcagcccc |
| 1261 | gacgtgcacg cgctgctgga gatcaccgag gagtcggacg ccgtgctggt ggacaagagc |
| 1321 | gacagcgact gagccgcccc cgctgccacc caccccattc ctcgctcctt ccgaacttcc |
| 1381 | tctttcgcat tctctctcgg ctcgagctgg ctgagatttt tctaaattga aaacacgccc |
| 1441 | ccctccccac acctccagga actccactcc cagtcttaga gctgttagga cccgatgggg |
| 1501 | aggcagcccc cgcagtggac aacccccgct tggacacagt ccgagtggaa tgggaaggga |
| 1561 | atggtcaatc cctgtcctgg ttgtccaagt cgggatctca gaggaaattg cagtgattcc |
| 1621 | acggttaggc ccccctgggg gggctgcctt cccctcagcc tctccccaca ccacccaccc |
| 1681 | agctgctgtc attccgctca ctgagctctt cttcattctc accctgatcc ctgggggact |
| 1741 | caaagccaaa actgcccaaa gaggaaagat tgaatcctaa aggggatccc tgcccccatg |
| 1801 | ggaggccccc tactagaagg acgtgaaagc agcttttggg ggaaactgag gcagtgggga |
| 1861 | agacagagca gaatgagccc tcaccctggc tgggggtcca gcacaggctg tatctgcaga |
| 1921 | gggtcccaga ggaacgctgg agccaagaga agccctggga aggaggggtg gggaacgaca |
| 1981 | tgcatgtgag ggatggcaca ctgatgtgtt tatgcacctg tacacaggag cgcatggcca |
| 2041 | tggctttgga aaggagaatg gaaaaataga agaaggtcgg ccgggcttgg tggcttatgc |
| 2101 | ctgtaacccc agcactttgg gaggccgagg tgggcggatc acctgaagtc aggagttcgg |
| 2161 | gaccagcctg gcaaacaccc catctctact aagcgaaaac ccatctctac taaaattaca |
| 2221 | aaaattagct gggcatggtt gcgcatgcct gtaatcccag ctactttgga ggctgaggtg |
| 2281 | gggagaattg cttgaacctg ggaggtggag gttgcagtga gccaaggtcg cgacactgca |
| 2341 | ctccagcctg ggtgacagag tgagactcca tctcaacaga aggaaaaaaa aggaaaatag |
| 2401 | gagaaggtgg aaatgggtga agagagaagt cccctcacta gctgcatgag aaatctatct |
| 2461 | tactgtggtt ctccatgggc agcaggacca tttttcagaa tcaagaggga ggacagtgtg |
| 2521 | agaaggcgat gatccaaaga agacagagag gtcagcccca cccgatccct caaatgggct |
| 2581 | cttggaggca cccccagggg cagcccattt ctcaaagtcc agaaaattag ggtcccagaa |
| 2641 | gggggcagca gcaggctggg agttaggagg gagagcaggg tgccggccct gccaccaagt |
| 2701 | tgagagctgg aggggaggtg gggagagaac atcacagagc agccagccct ggttcactcc |
| 2761 | tggcagtttc ttctcaagct ccttccctag gagcatggtg gcacgtgcct gtggtctcag |
| 2821 | ctacttggga ggctgaggcg ggaggatcgc cagagcccag gagtttgagg ctgcagtgag |
| 2881 | ctatgagggt gcctctgtgc tccatcctag gcaacagagt gatacgctgt ttaaaaagga |
| 2941 | aaaaatcctt ccctagagct agtatcctaa agctgcagag ctagcccaga cctcattggt |
| 3001 | ttccttgtcc ttggggtgct tttcctgaat ctttgcgggt gaagggagtg ttgctcccag |
| 3061 | tccagaggcc tgattctgtt tggactgggt tctcaagaca cgaccaggtt ctcaagacac |
| 3121 | gagtcccctt gttcctcccc attaaagggg gtttgtcaga agcaagaaca gcccctctcc |
| 3181 | ccagtcacag cctgaaggga ggccccgaga gcttcctcct tccccccacc tgctccttac |
| 3241 | cttctctgcc ctgcttttta gaactgcagt tcattgtttt aagggattgg gggagggagc |
| 3301 | ctggggacac aaacctttta tacaatacaa agctttgctt tttttttttt tttcttcctt |
| 3361 | ttccctttct cggttctctt ctctcctctg aatggctgaa gacccctctg ccgagggagg |
| 3421 | ttggggattg tgggacaagg tcccttggtg ctgatggcct gaaggggcct gagctgtggg |
| 3481 | cagatgcagt tttctgtggg cttggggaac ctctcacgtt gctgtgtcct ggtgagcagc |
| 3541 | ccgaccaata aacctgcttt tctaaaagga |
| CAVIN1 coding sequence | |
| (SEQ ID NO: 7; NM_012232.6) | |
| 1 | medptlyive rplpgypdae apepssagaq aaeepsgags eeliksdqvn gvlvlslldk |
| 61 | iigavdqiql tqaqleerqa emegavqsiq gelsklgkah attsntvskl lekvrkvsvn |
| 121 | vktvrgsler qagqikklev neaellerrn fkvmiyqdev klpaklsisk slkesealpe |
| 181 | kegeelgege rpeedaaale lssdeaveve evieesraer ikrsglrrvd dfkkafskek |
| 241 | mektkvrtre nlektriktk enlektrhtl ekrmnklgtr lvpaerrekl ktsrdklrks |
| 301 | ftpdhvvyar sktavykvpp ftfhvkkire gqvevlkate mvevgaddde ggaergeagd |
| 361 | lrrgsspdvh alleiteesd avlvdksdsd |

CAVIN1 amino acid sequence
(SEQ ID NO: 8; NP_036364.2)

1. A method of treatment of lipodystrophy comprising;
administering to an individual in need thereof a heterologous nucleic acid that encodes a therapeutic gene product,
wherein the lipodystrophy is characterised by a defective gene and the therapeutic gene product encoded by the heterologous nucleic acid is a functional version of the protein encoded by the defective gene or an RNA molecule that inhibits expression from the defective gene.

2. A method according to claim 1 wherein the amount of adipose tissue is increased in the individual following said administration.

3. A method according to claim 1 or claim 2 wherein the defective gene is an autosomal recessive gene and the heterologous nucleic acid encodes a functional version of the protein encoded by the defective gene

4. A method according to claim 3 wherein the lipodystrophy is a congenital generalised lipodystrophy (CGL).

5. A method according to claim 4 wherein the defective gene is selected from BSCL2, AGPAT2, CAV1, and CAVIN1.

6. A method according to claim 3 wherein the lipodystrophy is a familial partial lipodystrophy (FPLD).

7. A method according to claim 6 wherein the defective gene is CIDEC or LIPE.

8. A method according to claim 3 wherein the defective gene is selected from MFN2, PCTY1A, ZMPSTE24, and PSMB8

9. A method according to claim 1 or claim 2 wherein the defective gene is an autosomal dominant gene and the heterologous nucleic acid encodes an RNA molecule that inhibits expression of the defective gene

10. A method according to claim 9 wherein the lipodystrophy is a familial partial lipodystrophy (FPLD).

11. A method according to claim 10 wherein the defective gene is selected from LMNA, PPARG, PLIN1, AKT2 and ADRA2A.

12. A method according to claim 9 wherein the defective gene is selected from FBN1, POLD1 and PIK3R1.

13. A method according to any one of the preceding claims wherein the individual is an adult.

14. A method according to any one of the preceding claims wherein the individual lacks functional adipocytes.

15. A heterologous nucleic acid that encodes a therapeutic gene product for use in a method of treatment of lipodystrophy,
wherein the lipodystrophy is characterised by a defective gene and the therapeutic gene product encoded by the heterologous nucleic acid is a functional version of the protein encoded by the defective gene or an RNA molecule that inhibits expression from the defective gene.

16. A heterologous nucleic acid for use according to claim 15 wherein the method of treatment is a method according to any one of claims 1 to 14.

17. Use of heterologous nucleic acid that encodes a therapeutic gene product in the manufacture of a medicament for use in a method of treatment of lipodystrophy,
wherein the lipodystrophy is characterised by a defective gene and the therapeutic gene product encoded by the heterologous nucleic acid is a functional version of the protein encoded by the defective gene or an RNA molecule that inhibits expression from the defective gene.

18. Use according to claim 17 wherein the method of treatment is a method according to any one of claims 1 to 14.

19. A method according to any one of claims 1 to 14, a heterologous nucleic acid for use according to claim 15 or claim 16 or use according to claim 17 or claim 18 wherein the heterologous nucleic acid is contained in a viral vector.

20. A method, heterologous nucleic acid for use, or use according to claim 19 wherein the viral vector is an adenoviral vector.
